(19) United States
(12) Patent Application Publication
(10) Pub. No.: US 2021/0041378 A1
(43) Pub. Date: Feb. 11, 2021

(54) Systems and Methods for Using Three-Dimensional X-Ray Imaging in Meat Production and Processing Applications

(71) Applicant: Rapiscan Systems, Inc., Torrance, CA (US)

(72) Inventor: Edward James Morton, Guildford (GB)

(21) Appl. No.: 16/990,167

(22) Filed: Aug. 11, 2020

Related U.S. Application Data
(60) Provisional application No. 62/885,268, filed on Aug. 11, 2019.
(60) Provisional application No. 62/885,267, filed on Aug. 11, 2019.

(51) Int. Cl. G01N 23/046; G01N 23/083; G01N 23/18; G01N 33/483

(52) U.S. Cl. CPC G01N 23/046; G01N 23/083; G01N 23/18; G01N 33/4833; A61B 6/508; G01N 2223/20; G01N 2223/3307; G01N 2223/335; G01N 2223/501; G01N 2223/6126

(57) ABSTRACT

In embodiments, the present invention describes the use of three-dimensional (3D) stationary gantry X-ray computed tomography systems to scan animals/livestock for enabling improved management of animal farming processes, functions or events. The present invention also discloses the use of 3D stationary gantry X-ray computed tomography systems for carcass screening and improved abattoir production planning, execution, and automation. In various embodiments, use of the scanning technology supports high throughput, automated, meat-processing lines with reduced manual labor, objectively measured product quality and improved food safety standards. In embodiments, the present specification discloses the use of 3D X-ray inspection to generate an image of an entire carcass and sections of the carcass, during the stages of dissection, final product preparation, and packaging of the carcass.

### CROSS-REFERENCE TO RELATED APPLICATIONS

The present application relies on, for priority, U.S. Patent Provisional Application No. 62/885,267, entitled “Systems and Methods of Using 3D X-Ray Imaging in Meat Processing Factories”, filed on Aug. 11, 2019, and U.S. Patent Provisional Application No. 62/885,268, entitled “Systems and Methods of Using 3D X-Ray Imaging in Farms That Produce Livestock for Deriving Meat Products”, and filed on Aug. 11, 2019, both of which are herein incorporated by reference in their entirety.

### FIELD

The present specification relates generally to the field of rearing animals and/or livestock on farms for the processing and production of meat products derived therefrom. More specifically, the present specification is related to the use of three-dimensional (3D) stationary gantry computed tomography (CT) systems for improving farming practices that lead to enhanced quality of reared animal products in addition to improved management of abattoir production processes.

### BACKGROUND

Farms produce livestock destined for consumption in human and animal food chains, including but not limited to, poultry, pigs, goats, sheep and cattle. In contrast to other industries where a blending of product is possible to achieve a level of consistency, each animal has individual characteristics that warrant consumer satisfaction. The manner in which the animals are raised or treated on the farm tend to have an effect on the characteristics that affect customer satisfaction with meat products derived from the animals (such as, for example, a beefsteak or lamb chop). Consumers place increasing emphasis on consumption quality, food safety, and food traceability of the resultant meat product. As an example, animals reared at cattle farms are sold and processed at meat factories to produce a variety of meat products within the food chain. Strict quality control measures exist to ensure that the animals that enter the factory are optimally processed to produce products that meet desired consumer satisfaction in terms of eating quality, food chain traceability, and food safety.

To satisfy such consumer demands, the farmer needs to demonstrate conformance to standards and practices, in addition to regular farming activities, which place considerable burden on the farmer. The objective of a farmer is thus to breed the highest value animal for the farming conditions at a particular farm location (high altitude, low altitude, warm, cool, wet, dry, lush, barren) and to do this at the lowest possible cost. This means managing food, water, veterinary needs, transportation, and maintenance costs to deliver the greatest return. Currently, farmers use a range of information sources to plan their farming practices including weather forecasting, satellite imagery for pasture and water management, animal tracking to determine optimal location of feed and water troughs, genetic profiling for herd development and veterinary records. In general, such information is processed by the farmer using his own farming experience in order to optimize animal health, lean meat yield (the amount of meat compared to fat or bone), and consequent return on investment.

Once an animal reaches a meat processing plant or factory, the animals are typically slaughtered first; the head, viscera, hide and extremities are subsequently removed; and the carcasses are then placed into a cool room for a period of time to hang while fat solidifies. Once the carcass is rigid, it is then sectioned into major pieces (known as primals). Each primal is then passed on to a de-boning area in which retail ready cuts of meat are processed into bone-in or boneless cuts prior to packaging and transfer into the retail supply chain. Hundreds of people stand shoulder-to-shoulder to each perform a certain set of actions as the carcass or primal passes in front of them, with the carcass typically being suspended from a moving rail and the primal typically on a moving conveyor belt in this labor-intensive process. Instructions are provided to each individual in the de-boning area with regard to which cuts are required on each day to satisfy customer demand and meet production targets. The result is a productive process but not one that typically operates at peak efficiency.

Efficiency losses come from trimming excess meat off the retail cut, thus putting valuable product into a lower-grade food supply chain, for example overcutting valuable rib-eye muscle such that it ends up destined for lower value minced meat. Further efficiency losses come from inaccurate production planning in which a carcass is processed into a sub-optimal set of retail cuts. This typically occurs because the cutting team of individuals is provided with a production plan that is not specific to each individual carcass but rather reflects an average production target across the full set of carcasses to be processed that day.

Each individual working in the plant has an obligation to meet high standards of food safety, but in some cases, the carcass may contain invisible contamination or health defects that are hidden beneath the visible surface of the carcass that are not possible for the individual to determine. This can result in occasional, yet significant, food safety issues that can be expensive and complex to mitigate. Further, as retail cuts are produced and packaged, there are occasional errors in food labelling and packaging which result in shipping incorrect products to customers. Such errors lead to rejection, sometimes of large quantities, of product by retail customers or consumers. In these cases, there is an adverse financial impact on the processor and the rejected product usually needs to be destroyed. It should also be noted that meat processing plants or factories predominantly employ individual workers who use knives to stage-by-stage dissect a carcass into required consumer products. Thus, the individual workers in a meat processing line responsible for the slaughter of an animal all the way to the final packaging of a product must undergo a high level of training to achieve proper cutting technique on a repeatable basis at the processing line speed required to achieve a commercially satisfactory outcome.

In some sectors, the use of automation to either substitute for or augment the labor force is prevalent (for example, in poultry processing) but in other sectors, the use of automation is limited (for example, beef processing). In large part, this is driven by the complexity and variation of the anatomy between one carcass and another. In poultry, such variations are relatively minimal whereas in beef the variations can be large depending on the breed and weight of the carcass being processed.

On the retail end, customers of meat products have specific requirements for the quality and cut of the products that they buy from a meat factory. These may include meat grading, fat thickness, weight and other factors that the processor must conform to regardless of the supply of animals into the factory. Given that the processor only understands the actual anatomy of the carcass during the dissection process in the factory, it is hard to plan optimal production based on the significant variation in size, weight and quality of the animals that arrive at the factory. This may lead to directing higher quality product to lower value output streams thereby resulting in reduction in yield and factory efficiency.

Meat quality grading systems tend to rely on relatively subjective measurements of a carcass and may include characteristics such as, but not limited to: a) comparison of meat color to a standard color chart at a specific location in the carcass; b) comparison of marbling and fat content of the carcass compared to a set of standardized photographs; and c) the amount force needed to indent a particular point on the surface of the carcass among other subjective indicators. Such measurements tend to be point-based and do not measure the natural variation in meat quality that can occur either within a particular muscle group or between muscle groups.

There is therefore a need for use of X-ray scanning systems and methods to improve farming practices leading to a higher valuation of reared animals. There is also need for the use of X-ray screening at various stages of the animal life cycle during development on a farm so that meat products derived from a herd are better characterized in terms of food quality and food safety. There is also a need to improve production efficiency, to reduce labor utilization, to take a carcass-centric approach to production, to enhance plant and food safety performance and to reduce losses due to poorly labelled and poorly packaged product. Accordingly, there is need for use of X-ray scanning systems and methods for improved quality control, consumption quality, carcass valuation and food safety in meat processing factories or abattoirs. There is also need for the use of X-ray screening to aid overall production planning and automation for improved abattoir management.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a stationary gantry X-ray computed tomography (CT) imaging system adapted to scan an animal in a farm, wherein the CT imaging system is housed in a first enclosure and surrounded with at least one second enclosure and comprises: a horizontal platform configured to direct the animal through a scanning area for inspection; a first plurality of X-ray sources positioned at least partially around the scanning area to scan the animal in a first imaging plane; a first array of detectors, wherein the first array of detectors is offset from the associated first plurality of X-ray sources such that X-rays from each of the first plurality of X-ray sources on one side of the scanning area interact with corresponding each of the first array of detectors on an opposing side of the scanning area to form a first transmission image data of the animal; a second plurality of X-ray sources positioned at least partially around the scanning area to scan the animal in a second imaging plane; a second array of detectors, wherein the second array of detectors is offset from the associated second plurality of X-ray sources such that X-rays from each of the second plurality of X-ray sources on one side of the scanning area interact with corresponding each of the second array of detectors on an opposing side of the scanning area to form a second transmission image data of the animal; a controller configured to control an activation and deactivation of each of the first plurality of X-ray sources and each of the second plurality of X-ray sources; and at least one workstation configured to receive and process the first and second transmission image data and generate a three-dimensional image of the animal as the animal passes through the scanning area.

Optionally, the scanning area has a substantially rectangular geometry and a value representative of an entire width of the scanning area is within 85% of a value representative of an entire length of the scanning area.

Optionally, the first and second imaging planes are disposed along a direction perpendicular to the direction of motion of the animal over the horizontal platform.

Optionally, the first plurality of X-ray sources are offset from the associated first array of detectors, in the first imaging plane, by a first distance, the second plurality of X-ray sources are offset from the associated second array of detectors, in the second imaging plane, by a second distance, and the first distance is equal to the second distance and ranges from 2 mm to 20 mm.

Optionally, the first imaging plane comprises four X-ray sources separated from each other by gaps, and the second imaging plane comprises four X-ray sources positioned to align with the gaps.

Optionally, the first and second imaging planes are separated by a distance ranging from 200 mm to 2000 mm.

Optionally, the first plurality of X-ray sources and the second plurality of X-ray sources comprise linear multi-focus X-ray sources, and the controller is configured to switch on each source point within a first of the linear multi-focus X-ray sources and subsequently switch on each source point within a second of the linear multi-focus X-ray sources that is not adjacent to the first linear multi-focus X-ray source. Optionally, the second linear multi-focus X-ray source is 20 to 90 degrees away from the source point within the first linear multi-focus X-ray source.

The present specification also discloses a stationary gantry X-ray CT imaging system to scan an animal in a farm, comprising: a horizontal platform configured to enable the animal to pass through a scanning area for inspection; a plurality of X-ray sources disposed in a plane at least partially around the scanning area; an array of detectors deployed at least partially around the scanning area to form transmission scan data of the animal; a controller configured to control an activation and deactivation of each of the plurality of X-ray sources; and at least one workstation configured to received and process the transmission scan data and to determine at least one of lean meat yield, ratio of intra-muscular fat to tissue, amount of inter-muscular fat, absolute and relative size of individual organs, and presence of cysts, tumors, pleurisy and foreign objects corresponding to the animal.

Optionally, the plurality of X-ray sources comprises 200 to 500 X-ray source emission points around an anode and each of the X-ray source emission points and the anode are enclosed in a vacuum tube.

Optionally, each of the X-ray source emission points are characterized by a tube voltage in a range of 120 kV to 200 kV and a tube current in a range 1 mA to 20 mA.

Optionally, each of the plurality of the X-ray sources is configured to be operated at a tube voltage of 160 kV and at a tube current of 4 mA. Optionally, each of the plurality of the X-ray sources is operated corresponding to total X-ray beam power of 640 W.

Optionally, each of the plurality of the X-ray sources comprises an X-ray tube.

Optionally, the plurality of X-ray sources are adapted to be operated to deliver a dose per scan to the animal in a range of 2 μSv to 20 μSv.

Optionally, each of the plurality of X-ray sources are offset from the array of detectors by a distance ranging from 2 mm to 20 mm.

The system may further comprise a sensor adapted to monitor a surface profile of the animal and measure a motion of the animal. Optionally, the controller is configured to use the measured motion of the animal to determine where X-ray projections should be back-projected into a three-dimensional reconstructed image volume.

The system may further comprise a first inclined ramp adapted to enable the animal to pass into the scanning area and a second inclined ramp adapted to enable the animal to pass out of the scanning area.

The present specification also discloses a stationary gantry X-ray CT imaging system to scan an animal in a farm, comprising: a horizontal platform configured to receive the animal passing through a scanning area for inspection; at least one X-ray source disposed in a plane around the scanning area; an array of detectors deployed around the scanning area to form transmission scan data of the animal as a result of interaction of X-rays from said at least one X-ray source; a controller configured to control an activation and deactivation of the at least one X-ray source; and a radar imaging system comprising a plurality of transceivers adapted to determine a shape and a movement of the animal passing through the scanning area, wherein the plurality of transceiver cards are disposed on a first vertical side of the scanning area and a second vertical side of the scanning area, and wherein each transceiver card comprises a plurality of transmitter and receiver elements; and at least one workstation configured to receive and process the transmission scan data from the array of detectors and to receive and process data indicative of the shape and the movement of the animal from the radar imaging system and further configured to determine at least one of lean meat yield, ratio of intra-muscular fat to tissue, amount of inter-muscular fat, absolute and relative size of individual organs, and presence of cysts, tumors, pleurisy and foreign objects corresponding to the animal.

Optionally, the radar imaging system is adapted to be operated in a stepped frequency continuous wave radar scanning mode.

Optionally, each of the plurality of transceiver cards comprises 8 receiver and 8 transmitter elements.

Optionally, each transmitter element is activated with all receiver elements listening in parallel to form a tomographic data set. Optionally, the radar imaging system is configured to reconstruct the tomographic data to from a surface image of the animal.

Optionally, each transmitter element is held at a discrete set of frequencies with steps and a fixed period of time per step.

The present specification also discloses a method of using a plurality of three-dimensional X-ray computed tomography scanning processes for scanning animals in a farm through various points in a lifecycle of an animal, the method comprising: while an animal is in a first age range, obtaining a first scan of the animal, using data from the first scan to identify abnormalities and determine predefined genetic features of the animal, and recording the abnormalities and the predefined genetic features; while an animal is in a second age range, obtaining a second scan of the animal, using data from the second scan to determine any abnormalities and health conditions of the animal, and recording the abnormalities and health conditions; while an animal is in a third age range, obtaining a third scan of the animal using data from the third scan for determining a quality of the animal and a herd to which the animal belongs for evaluating a value of the animal and the herd; and before the animal reaches an age of M, obtaining a fourth scan of the animal the animal is ready for auction.

Optionally, data from said third and fourth scans is used to determine a value of the animal based on at least one of a plurality of pre-sale parameters, said plurality of pre-sale parameters including lean meat yield, ratio of intra-muscular fat to tissue, amount of inter-muscular fat, absolute and relative size of individual organs, muscle volume, number of ribs, and presence or absence of cysts, tumors, pleurisy and foreign objects. The method may further comprise obtaining a fifth scan of the animal following sale of the animal from the farm for determining one or more of a plurality of after-sale parameters and comparing a least a portion of the plurality of after-sale parameters with at least a portion of the plurality of pre-sale parameters, wherein the plurality of after-sale parameters include lean meat yield, ratio of intra-muscular fat to tissue, amount of inter-muscular fat, absolute and relative size of individual organs, muscle volume, number of ribs, and presence or absence of cysts, tumors, pleurisy and foreign objects.

The present specification also discloses a stationary gantry X-ray CT imaging system to scan carcasses in an abattoir, said system being housed in a first enclosure and at least partially enclosed with a shielding tunnel and comprising: a conveyor rail to move the carcasses through an inspection area at a predefined speed, wherein the carcasses hang from hooks of the conveyor rail; a first plurality of X-ray sources positioned around the inspection area to scan the carcasses in a first imaging plane; a first array of detectors, wherein the first array of detectors is offset from the associated first plurality of X-ray sources such that X-rays from each of the first plurality of X-ray sources on one side of the inspection area interact with corresponding each of the first array of detectors on an opposing side of the inspection area to form a first transmission image through each of the carcasses; a second plurality of X-ray sources positioned around the inspection area to scan the carcasses in a second imaging plane; a second array of detectors, wherein the second array of detectors is offset from the associated second plurality of X-ray sources such that X-rays from each of the second plurality of X-ray sources on one side of the inspection area interact with corresponding each of the second array of detectors on an opposing side of the inspection area to form a second transmission image through each of the carcasses; and at least one workstation configured to process the first and second transmission images and generate a three dimensional image of each of the carcasses.

Optionally, the predefined speed ranges from 0.05 m/s to 0.5 m/s.

Optionally, the first and second imaging planes are along a direction perpendicular to the direction of motion of the carcasses along the conveyor rail.

Optionally, the first plurality of X-ray sources are offset from the associated first array of detectors, in the first imaging plane, by a first distance, the second plurality of linear X-ray sources are offset from the associated second array of detectors, in the second imaging plane, by a second distance, and the first distance is equal to the second distance.

Optionally, the shielding tunnel and the conveyor rail are configured such that there is no straight path through the shielding tunnel and the conveyor rail and that any path through the shielding tunnel and the conveyor rail requires at least one turn having a turn radius greater than 10%.

Optionally, the shielding tunnel and the conveyor rail have a linear layout but with one or more chicanes.

Optionally, the first imaging plane comprises five X-ray sources separated from each other by gaps, and the second imaging plane comprises five X-ray sources positioned to fill said gaps.

Optionally, the inspection area has a cross-sectional area defined by a width that is less than 40% of a height.

Optionally, the first and second imaging planes are separated by a distance of approximately 500 mm.

The present specification also discloses a stationary gantry X-ray CT imaging system to scan carcasses in an abattoir, said system being housed in a first enclosure and at least partially surrounded with at least one radiation shielding tunnel and comprising: a conveyor rail to move the carcasses through an inspection area at a predefined speed, wherein the carcasses hang from hooks of the conveyor rail, and wherein the inspection area has a polygonal shape; a first plurality of X-ray sources positioned around the inspection area to scan the carcasses in a first imaging plane, wherein the first imaging plane comprises a first number of X-ray sources separated from each other by gaps; a first array of detectors, wherein the first array of detectors is offset from the associated first plurality of X-ray sources such that X-rays from each of the first plurality of X-ray sources on one side of the inspection area interact with corresponding each of the first array of detectors on an opposing side of the inspection area to form a first transmission scan data through each of the carcasses; a second plurality of X-ray sources positioned around the inspection area to scan the carcasses in a second imaging plane, wherein the second imaging plane comprises first number of X-ray sources positioned to fill the gaps; a second array of detectors, wherein the second array of detectors is offset from the associated second plurality of X-ray sources such that X-rays from each of the second plurality of X-ray sources on one side of the inspection area interact with corresponding each of the second array of detectors on an opposing side of the inspection area to form a second transmission scan data through each of the carcasses; and at least one workstation configured to process the first and second transmission scan data and determine at least one of lean meat yield, ratio of intra-muscular fat to tissue, amount of inter-muscular fat, absolute and relative size of individual organs, muscle volume, number of ribs, and presence of cysts, tumors, pleurisy and foreign objects corresponding to each of the carcasses.

Optionally, the predefined speed ranges from 0.05 m/s to 0.5 m/s.

Optionally, the first and second imaging planes are along a direction perpendicular to the direction of motion of the carcasses along the conveyor rail.

Optionally, the first plurality of X-ray sources are offset from the associated first array of detectors, in the first imaging plane, by a first distance, the second plurality of X-ray sources are offset from the associated second array of detectors, in the second imaging plane, by a second distance, and the first distance is equal to the second distance.

Optionally, the shielding tunnel and the conveyor rail are configured such that there is no straight path through the shielding tunnel and the conveyor rail and that any path through the shielding tunnel and the conveyor rail requires at least one turn having a turn radius greater than 10%.

Optionally, the shielding tunnel and the conveyor rail have a layout in a broadly linear fashion with one or more chicanes.

Optionally, the inspection area has a cross-sectional area defined by a maximum width that is less than 20% of a maximum height.

Optionally, the first and second imaging planes are separated by a distance of approximately 500 mm.

The present specification also discloses a stationary gantry X-ray CT imaging system to scan carcasses in an abattoir, comprising: a conveyor rail to move the carcasses through an inspection area at a speed ranging from 0.05 m/s to 0.5 m/s, wherein the carcasses hang from hooks of the conveyor rail; a first plurality of linear multi-focus X-ray sources positioned around the inspection area to scan the carcasses in a first imaging plane, wherein the first imaging plane comprises ‘m’ linear multi-focus X-ray sources separated from each other by gaps; a first array of detectors, wherein the first array of detectors is offset from the associated first plurality of linear multi-focus X-ray sources such that X-rays from each of the first plurality of multi-focus X-ray sources on one side of the inspection area interact with corresponding each of the first array of detectors on an opposing side of the inspection area to form a first transmission scan data through each of the carcasses; a second plurality of linear multi-focus X-ray sources positioned around the inspection area to scan the carcasses in a second imaging plane, wherein the second imaging plane comprises ‘n’ linear multi-focus X-ray sources positioned to fill the gaps, and wherein the first and second imaging planes are along a direction perpendicular to the direction of motion of the carcasses along the conveyor rail; a second array of detectors, wherein the second array of detectors is offset from the associated second plurality of linear multi-focus X-ray sources such that X-rays from each of the second plurality of multi-focus X-ray sources on one side of the inspection area interact with corresponding each of the second array of detectors on an opposing side of the inspection area to form a second transmission scan data through each of the carcasses; and at least one workstation configured to process the first and second transmission scan data and determine at least one of production planning, eating quality and health corresponding to each of the carcasses.

Optionally, m=n=five, and the inspection area has a cross-sectional area of 1500 mm (width)×3900 mm (height).

Optionally, m=n=three, and the inspection area has a cross-sectional area of 1500 mm (width)×2000 mm (height).

The present specification also discloses a method of processing meat in an abattoir of a meat processing plant by using a stationary gantry X-ray CT imaging system, said method comprising: scanning full carcass of an animal after removal of skin, offal, extremities and trim waste, wherein the scanning is performed while the carcass has a temperature between 10 degrees Celsius and 50 degrees Celsius for obtaining scan data to determine a value of the carcass based on at least one of lean meat yield, ratio of intra-muscular fat to tissue, amount of inter-muscular fat, absolute and relative size of individual organs, muscle volume, number of ribs, and presence of cysts, tumors, pleurisy and foreign objects; scanning the offal, extremities and trim waste removed from the animal for obtaining scan data to determine organ abnormalities and presence or absence of cysts, tumors, pleurisy and foreign objects; storing the carcass, for cooling, at a temperature less than 15 degrees Celsius; using an X-ray system, scanning the carcass after cooling for a predefined period of time; using a controller, obtaining scan image data from the X-ray system and generating data indicative of areas of contiguous meat of a predefined quality level; and transmitting said data to an automated cutting system, wherein the automated cutting system is adapted to use said data to segment the carcass into pieces.

The method may further comprise: packaging said pieces; scanning the packaged pieces for foreign objects and fat thickness; boxing the scanned packaged pieces; and scanning the boxed packaged pieces to validate that the boxed package has a predefined number of pieces of a predefined type, eating quality, shape and size.

Optionally, the eating quality is determined based on at least one of a ratio of intra-muscular fat to tissue and an amount of inter-muscular fat.

Optionally, the predefined period of time ranges from 24 to 36 hours.

Optionally, the automated cutting system performs deboning of the carcass by using the scanned images of the cooled carcass. The method may further comprise determining an amount of meat remaining on a bone of the carcass after de-boning and repeating the deboning if the amount of meat is more than a predefined amount.

The present specification also discloses a method of automating a process of meat production in a meat production plant by using a plurality of sensing and imaging devices coupled with a computer network running a plurality of analysis algorithms on data obtained by the sensing and imaging devices, said data being at least temporarily stored in a database coupled with the computer network, the method comprising: scanning an animal carcass by using the sensing and imaging devices to obtain data with respect to at least health of the animal and quality of the carcass; storing said data in the database; processing said data by using the plurality of analysis algorithms to obtain parameters for controlling production of pieces of meat from said carcass, wherein each of the pieces has a predefined shape, size, weight and quality; and using the processed data for planning meat production in the meat production plant.

Optionally, the sensing and imaging devices comprise one or more of 3D X-Ray tomographic scanners, 2D X-Ray tomographic scanners, hyperspectral and fluorescence scanners, handheld sensing devices, 3D X-Ray scanners, 2D X-Ray scanners RFID readers, barcode readers, and cameras.

Optionally, the computer network is coupled to a plurality of inspection workstations for communicating with operators of the plant the data processing and analysis parameters.

Optionally, the analysis algorithms comprise at least one of meat grading algorithms, carcass valuation algorithms, production planning algorithms, animal health algorithms, and product quality check and validation algorithms.

Optionally, the computer network is further coupled with one or more quality control systems and automation systems.

The method may further comprise using the production controlling parameters in real-time to operate an automated cutting system of the meat production plant for guiding cutting of carcasses and primals into retail cuts.

The method may further comprise using the production controlling parameters in real-time to be analyzed by automated quality control processes and human quality control staff of the plant to ensure accurate processing and food safety standards.

Optionally, the inspection workstation operates as a plant management dashboard providing an operator with real-time updates of the status of all products and staff within the plant, said status real-time location of a carcass, primal, retail-cut, trim or packaged product identified by means of a unique ID.

The method may further comprise using the production controlling parameters in real-time to be analyzed to determine distinguishing characteristics of high performing operators, and using said determined distinguishing characteristics for training low performing operators of the plant.

Optionally, the hyperspectral and fluorescence scanners operate across the mid infra-red wavelengths ranging from 5,000 nm to 2,000 nm, short wave infra-red wavelengths ranging from 2,000 nm to 900 nm, near infra-red wavelengths ranging from 900 nm to 800 nm, visible light wavelengths ranging from 800 um to 400 nm and ultra-violet wavelength ranging from 400 nm to 100 nm, said hyperspectral and fluorescence scanners illuminating meat products under inspection for reflective image formation and analysis to detect one or more defects in said products.

Optionally, the cameras are video cameras operating in the visible wavelength ranging from 800 nm to 400 nm, and short wave infra-red wavelength region ranging from 2000 nm to 900 nm, said cameras being used for tracking meat products and operating staff throughout the plant.

The method may further comprise assigning a unique identification code to each meat product comprising each of the carcasses being processed in the meat processing plant, each of the primals that are cut from said carcasses and each subsequent retail cut from each of said primals for tracking each of said products throughout the plant.

The present specification also discloses a method of assigning a carcass ID for tracking a location and time or arrival of each carcass through a meat processing plant by using a plurality of sensing and imaging devices coupled with a computer network running a plurality of analysis algorithms on data obtained by the sensing and imaging devices, said data being at least temporarily stored in a database coupled with the computer network; the method comprising: associating each abattoir hook of the plant with an RFID tag, wherein said hooks being used for suspending animal carcass over a moving rail; associating each animal arriving at the plant with an RFID ear tag; suspending each slaughtered animal carcass on an abattoir hook; and generating a unique ID for each carcass suspended on a hook by associating the hook RFID tag with the RFID tag of the animal corresponding to the carcass.

The method may further comprise tracking a carcass throughout the plant by using the unique carcass ID.

The present specification also discloses a method of tracking a primal cut from an animal carcass at a first location in a meat processing plant and moved through multiple locations in the plant by using at least a plurality of video cameras coupled with a computer network running a plurality of analysis algorithms on data obtained by the video cameras, said data being at least temporarily stored in a database coupled with the computer network; the method comprising: recording when a primal is first cut from the animal carcass at the first location; assigning a unique ID to the cut primal; tracking the primal by using the ID in real time to determine when the primal is placed at a second location by using at least one video camera; determining if the primal is fixed to a hook of an abattoir of the plant; associating the primal ID with an RFID of the hook RFID if the primal is fixed to the hook; determining if remains of the primal are removed from the hook; transferring the primal ID to a subsequent conveyor or waste chute of the meat processing plant, if remains of the primal are removed from the hook; determining if the primal is placed on a conveyor of the plant; associating the primal ID with an adjacent RFID tag embedded in the conveyor; determining if the primal is transferred from the conveyor to a second conveyor of the plant; and automatically transferring the primal ID directly from the conveyor to the second conveyor, by using video cameras if the primal is transferred from the conveyor to the second conveyor.

Optionally, conveyor ID tags are placed at a spacing ranging from 100 mm to 200 mm on the conveyor for making the position of the primal identifiable by the video cameras.

The method may further comprise tracking more than one primal placed on a conveyor, wherein a plurality of primals are associated with the same conveyor RFID tag, by using video cameras to determine a lateral position of each primal on the conveyor at a location in the plant where the primals are loaded or removed from the conveyor.

The present specification also discloses a method of tracking a location of one or more pieces of meat cut from an animal carcass by human operators through a meat processing plant and moved through multiple locations in the plant by using at least a plurality of video cameras coupled with a computer network running a plurality of analysis algorithms on data obtained by the video cameras, said data being at least temporarily stored in a database coupled with the computer network; the method comprising: placing one or more pieces of meat cut from the animal carcass in a trim bin, wherein each of said piece is assigned a unique ID; recording the unique IDs of each piece placed in the trim bin against a unique RFID of the bins; aggregating the pieces placed in multiple trim bins in one large bin; associating a unique RFID of the large bin with the unique RFIDs of the multiple trim bins; and associating a unique RFID of the large bin with the unique IDs of each piece placed in said multiple trim bins for associating each meat piece placed in the large bin with the animal carcass.

The method may further comprise deleting from the database all IDs associated with the multiple trim bins and the IDs of the pieces placed in the large bin after the large bin is emptied.

The aforementioned and other embodiments of the present specification shall be described in greater depth in the drawings and detailed description provided below.

### BRIEF DESCRIPTION OF THE DRAWINGS

These and other features and advantages of the present specification will be further appreciated, as they become better understood by reference to the following detailed description when considered in connection with the accompanying drawings:

FIG. 1A shows a first cross-sectional side view of a 3D stationary gantry X-ray CT imaging system configured to scan cattle at farms, in accordance with some embodiments of the present specification;

FIG. 1B shows a second cross-sectional side view of the 3D stationary gantry X-ray CT imaging system of FIG. 1A, in accordance with some embodiments of the present specification;

FIG. 1C shows a 3D stationary gantry X-ray CT imaging system comprising a plurality of X-ray tubes, in accordance with some embodiments of the present specification;

FIG. 2 shows bottom, top, longitudinal side and end views of a linear multi-focus X-ray source for use in a 3D stationary gantry X-ray CT imaging system, in accordance with some embodiments of the present specification;

FIG. 3A shows first side, second side and top views of a single-plane stationary gantry X-ray computed tomography system configured to scan cattle at farms, in accordance with some embodiments of the present specification;

FIG. 3B shows the first side view of the single-plane stationary gantry X-ray computed tomography system of FIG. 3A including a radar imaging or inspection system, in accordance with some embodiments of the present specification;

FIG. 4 illustrates an exemplary stepped frequency continuous wave radar scanning sequence, in accordance with some embodiments of the present specification;

FIG. 5 is a block diagram of a radar imaging system, in accordance with some embodiments of the present specification;

FIG. 6 shows an exemplary arrangement of a plurality of transmitter (Tx) and receiver (Rx) elements of a radar imaging or inspection system, in accordance with some embodiments of the present specification;

FIG. 7 is a block diagram of a plurality of exemplary information, outputs or outcomes derived based on processing or analyses of an animal's scan image data generated using a 3D stationary gantry X-ray CT imaging system, in accordance with some embodiments of the present specification;

FIG. 8 is a workflow illustrating use of a plurality of 3D X-ray computed tomography scanning processes during various events relating to farming of livestock, in accordance with some embodiments of the present specification;

FIG. 9A illustrates a top view of a 3D stationary gantry X-ray CT imaging system in first configuration to scan meat in an abattoir, in accordance with some embodiments of the present specification;

FIG. 9B illustrates a top view of the 3D stationary gantry X-ray CT imaging system of FIG. 1A in second configuration to scan meat in an abattoir, in accordance with some embodiments of the present specification;

FIG. 10A illustrates first, second and third cross-sectional views of a 3D stationary gantry X-ray CT imaging system configured for dual-plane scanning of carcasses, in accordance with some embodiments of the present specification;

FIG. 10B illustrates a fourth cross-sectional view of the 3D stationary gantry X-ray CT imaging system 200, in accordance with some embodiments of the present specification;

FIG. 11 illustrates first, second and third cross-sectional views of a 3D stationary gantry X-ray CT imaging system configured for dual-plane scanning of carcasses, in accordance with some embodiments of the present specification;

FIG. 12 illustrates a cross-sectional view of a 3D stationary gantry X-ray CT imaging system configured for single-plane scanning of carcasses, in accordance with some embodiments of the present specification;

FIG. 13 shows bottom, top, longitudinal side and end views of a linear multi-focus X-ray source for use in a 3D stationary gantry X-ray CT imaging system, in accordance with embodiments of the present specification;

FIG. 14 is a block diagram illustration of a plurality of exemplary information, outputs or outcomes derived based on processing of carcass scan image data generated using a dual-plane 3D stationary gantry X-ray CT imaging system, in accordance with some embodiments of the present specification;

FIG. 15 is a workflow illustrating use of a plurality of 3D X-ray computed tomography scanning processes for improved abattoir management and automation, in accordance with some embodiments of the present specification;

FIG. 16A is a workflow illustrating a semi-automated meat production process, in accordance with an embodiment of the present specification;

FIG. 16B is a block diagram illustrating an augmented reality based system for cutting meat in a meat processing plant, in accordance with an embodiment of the present specification;

FIG. 16C is a flowchart illustrating the steps of an augmented reality based method for cutting meat in a meat processing plant, in accordance with an embodiment of the present specification;

FIG. 17 is a flowchart illustrating the steps of assigning a carcass ID for tracking a location, time and/or arrival of each carcass through a meat processing plant, in accordance with an embodiment of the present specification;

FIG. 18 is a flowchart illustrating the steps of assigning a carcass ID for tracking a location and/or time when a primal or retail cut is obtained from a carcass through a meat processing plant, in accordance with an embodiment of the present specification; and

FIG. 19 is a flowchart illustrating the steps of assigning a carcass ID for tracking a location of a carcass/primal/retail cut through a meat processing plant, in accordance with an embodiment of the present specification.

### DETAILED DESCRIPTION

In an embodiment, the present specification describes the use of three-dimensional (3D) stationary gantry X-ray computed tomography systems to scan animals and/or livestock for enabling improved management of animal farming processes, functions, or events. The resultant scan information, particularly when generated or applied at different stages during the development of an animal, may be used to drive farming practices for individual animals and for overall development of one or more herds. When such farming practices are driven based on scan information of animals and herds, the result is improved valuation of animals, a reduction in farming costs, and a concurrent improvement in eating or consumption quality of each animal thereby leading to improved farm economics and consumer satisfaction.

The present specification also discloses the use of 3D stationary gantry X-ray computed tomography systems for carcass screening and improved abattoir production planning, execution, and automation. In various embodiments, the use of scanning technology supports high throughput, automated, meat-processing lines with reduced manual labor, objectively measured product quality and improved food safety standards.

In an embodiment, the present specification discloses the use of 3D X-ray inspection to generate an image of an entire carcass and sections of the carcass, during the stages of dissection, final product preparation, and packaging of the carcass. The generated images are used to derive metrics on, but not limited to, eating quality, animal health, lean meat yield (the amount of meat, fat and bone present in the carcass), carcass value, and 3D carcass structure. The derived metrics also drive abattoir efficiency through process automation, precise production planning, provision of accurate consumption quality through each muscle within the carcass, rejection of unhealthy carcasses from the food chain, payment based on carcass value and not just on weight, quality control measures to ensure integrity of safe product to consumers, and supply chain assurance for customers to validate the supply chain of the meat that they purchase.

In an embodiment, the present specification also discloses a method for automating and increasing the efficiency of meat production in a meat processing plant. In an embodiment, the present specification provides for the use of network connected 2D and 3D X-ray imaging modalities along with visible and hand-held sensors such as, but not limited to, RFID and barcode readers in a meat producing plant. The networked imaging and screening modalities are used to generate data that is processed in real-time by specific algorithms in conjunction with production requirement information stored in a database that is coupled with the network, to generate individualized carcass-driven optimization of the meat production process as a whole. In an embodiment, the present specification provides a method for automatic and robotic cutting of carcasses.

In various embodiments, a computing device includes an input/output controller, at least one communication interface and a system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across a network of multiple computer systems and architectures. In some embodiments, execution of a plurality of sequences of programmatic instructions or code, which are stored in one or more non-volatile memories, enable or cause the CPU of the computing device to perform various functions and processes such as, for example, performing tomographic image reconstruction for display on a screen. In alternate embodiments, hard-wired circuitry may be used in place of, or in combination with, software instructions for implementation of the processes of systems and methods described in this application. Thus, the systems and methods described are not limited to any specific combination of hardware and software.

The term “pass”, “passes”, “passes through”, “passing through”, or “traverses” used in this disclosure encompass all forms of active and passive animal movement, including walking, being carried in a container, hanging from a structure or being conveyed/driven using a conveyor.

The term “meat” used in this disclosure may refer to flesh of animals used for food. In some embodiments, “meat” may refer to flesh inclusive of bone and edible parts but exclusive of inedible parts. Edible parts may include prime cuts, choice cuts, edible offals (head or head meat, tongue, brains, heart, liver, spleen, stomach or tripes and, in some cases, other parts such as feet, throat and lungs). Inedible parts may include hides and skins (except in the case of pigs), as well as hoofs and stomach contents.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. In addition, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

As used herein, the indefinite articles “a” and “an” mean “at least one” or “one or more” unless the context clearly dictates otherwise.

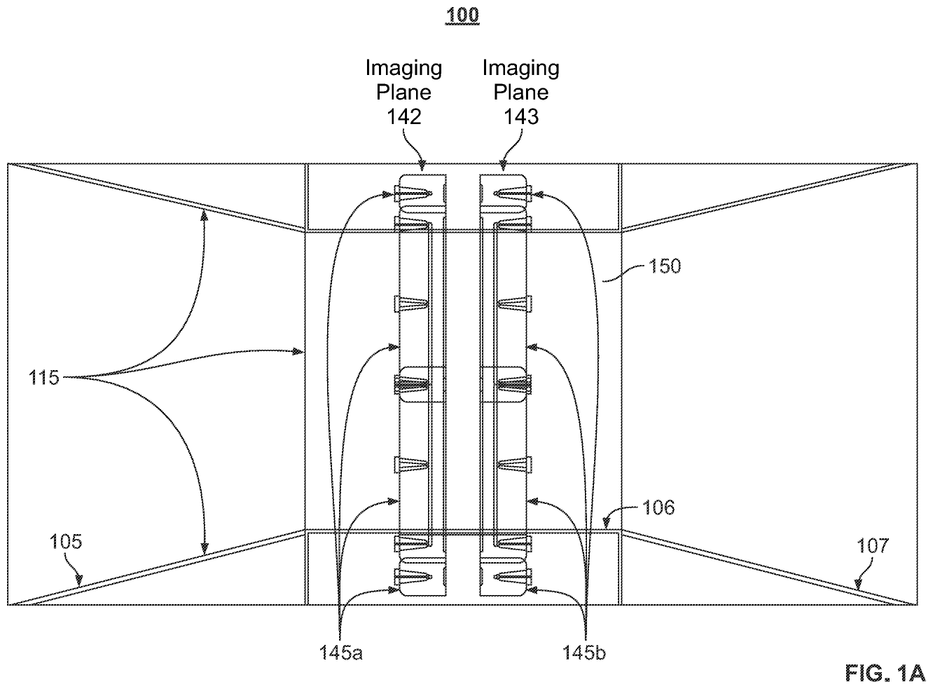

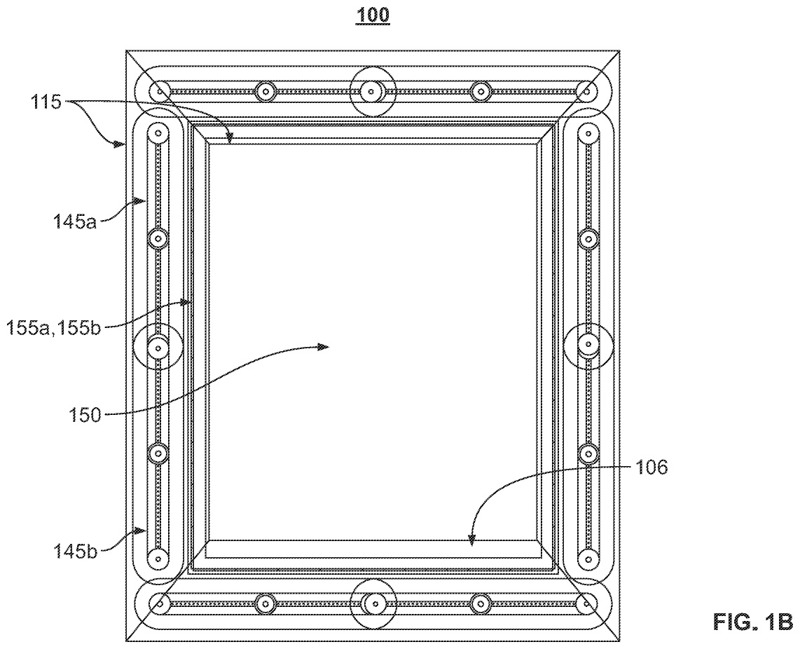

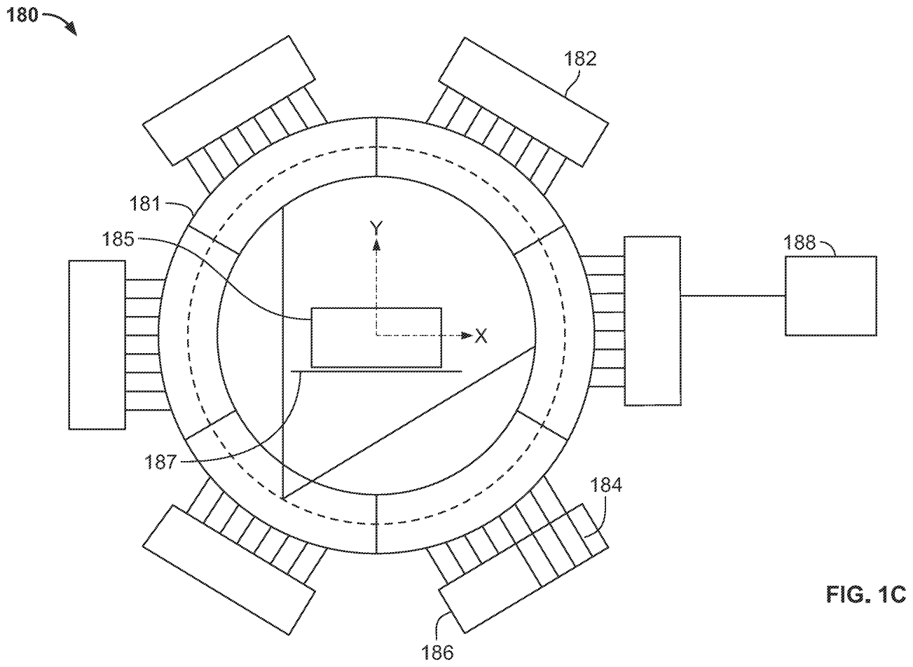

FIGS. 1A and 1B illustrate first and second side cross-sectional views of a 3D stationary gantry X-ray CT imaging system 100 (also referred to as a Real-Time Tomography (RTT) system) configured to scan cattle, in accordance with some embodiments of the present specification. Referring to FIGS. 1A and 1B, the system 100 is deployed, for example, in an animal farm to scan cattle in real time as an animal passes through a scanning region, area or aperture 150 of the system 100. The first side cross-sectional view of FIG. 1A is in a direction perpendicular to the direction of motion of an animal as it passes through the scanning region, area or aperture 150 whereas the second side cross-sectional view of FIG. 1B is in a direction parallel to the direction of motion of the animal passing through the scanning region, area or aperture 150.

In some embodiments, a first inclined ramp 105 is adapted to enable the animal to pass onto a horizontal platform 106 that lies in the scanning region, area or aperture 150 to eventually pass down using a second inclined ramp 107. In other words, the animal enters the scanning region, area or aperture 150 from the left portion in the figure and exits the scanning region, area or aperture 150 at the right in the figure.

In some embodiments, the system 100 is enclosed within a food safe, environmentally protected enclosure 115 manufactured using materials such as, but not limited to, stainless steel and/or plastic. In some embodiments, the system 100 is surrounded with at least one radiation shielding enclosure. A control room is provided for one or more system operators to review the performance of the system 100 on one or more inspection workstations in data communication with the system 100. In various embodiments, the one or more inspection workstations are computing devices.

In some embodiments, the system 100 is configured for dual-plane scanning and comprises a first plurality of linear multi-focus X-ray sources 145a along with an associated first array of detectors 155a positioned or deployed around the scanning region, area or aperture 150 to scan the animal in a first imaging plane 142 and a second plurality of linear multi-focus X-ray sources 145b along with an associated second array of detectors 155b also positioned or deployed around the scanning region, area or aperture 150 to scan the animal in a second imaging plane 143. Thus, the system 100 is constructed in two separate planes 142, 143 with data combined together, at the one or more inspection workstations, to create a single reconstructed volume.

In some embodiments, the scanning region, area or aperture 150 has a substantially rectangular geometry or shape. In some embodiments, a value representative of an entire width of the scanning area 150 is within 85% of a value representative of an entire height of the scanning area 150. In some embodiments, the scanning region, area or aperture 150 has dimensions 1500 mm (width)×1800 mm (height). In alternate embodiments, the scanning region, area or aperture 150 has a substantially square or polygonal geometry or shape. In some embodiments, the first imaging plane 142 comprises, say, four linear multi-focus X-ray sources 145a separated from each other and positioned around or along a perimeter of the scanning region, area or aperture 150. In some embodiments, the second imaging plane 143 comprises, say, four linear multi-focus X-ray sources 145b separated from each other and positioned around or along the perimeter of the scanning region, area or aperture 150.

In some embodiments, as shown in FIG. 1B, the linear multi-focus X-ray sources 145b (in the second imaging plane 143) are disposed or positioned so as to fill the gaps separating the linear multi-focus X-ray sources 145a (in the first imaging plane 142). Thus, the first and second linear multi-focus X-ray sources 145a, 145b are dispersed in their respective first and second imaging planes 142, 143 to create a substantially uniform sampling distribution around the periphery of the scanning region, area or aperture 150. In embodiments, it is preferred to maintain a relatively thin X-ray window around the X-ray detector regions 155a, 155b. In some embodiments, the horizontal top as well as first and second vertical sides use 2 mm to 5 mm thick aluminum. In the floor (horizontal platform 106), a thicker plate is needed, with a thickness ranging from 6 mm to 10 mm aluminum to prevent deformation under load from an animal's hoof Although such thick windows reduce the total X-ray flux in the scanning region 150, this also reduces low energy X-ray dose, which helps to reduce radiation dose to the animal to a tolerable level.

In some embodiments, the first and second imaging planes 142, 143 are disposed along a direction perpendicular to the direction of motion of the animal over the horizontal platform 106 and through the inspection region, area or aperture 150 during scanning. In embodiments, the first and second imaging planes 142, 143 are separated from each other, along the direction of motion of the animal during scanning, by a distance ‘d’ ranging from 100 mm to 2000 mm. Thus, the first plurality of linear multi-focus X-ray sources 145a and the associated first array of detectors 155a are deployed in the first imaging plane 142 while the second plurality of linear multi-focus X-ray sources 145b and the associated second array of detectors 155b are deployed in the second imaging plane 143.

In embodiments, the first plurality of linear multi-focus X-ray sources 145a are offset or displaced from the associated first array of detectors 155a, in the first imaging plane 142, by a distance d₁ while the second plurality of linear multi-focus X-ray sources are offset or displaced from the associated second array of detectors 155b, in the second imaging plane 143, by a distance d₂. In some embodiments, d₁ is equal to d₂. In various embodiments, the distances d₁ and d₂ range from 2 mm to 20 mm. It should be appreciated that the first and second array of detectors 155a, 155b are displaced from the respective planes of the first and second X-ray sources 145a, 145b so that X-rays from a source on one side of the scanning region, area or aperture 150 pass above the detector array adjacent to the source but interact in the detector array opposite to the source at the other side of the scanning region, area or aperture 150.

In an embodiment, the 3D stationary gantry X-ray CT imaging system 100 comprises a series of X-ray tubes operating in tandem, instead of a multi focus X-ray source shown in FIGS. 1A and 1B. In other words, the X-ray sources are a plurality of X-ray tubes and do not contain multiple source points.

In some embodiments, as shown in FIG. 1C, the 3D stationary gantry X-ray CT imaging system 180 comprises one or more X-ray tubes 181 which are configured into a substantially circular arrangement around the scanner axis, wherein each X-ray tube 181 contains an X-ray source having one or more X-ray source points 182. In an embodiment, the emission of X-rays from each source point from each of the X-ray tubes 181 is controlled by a switching circuit 184, with one independent switching circuit for each X-ray source point. The switching circuits for each tube 181 together form part of a control circuit 186 for that tube. A controller 188 controls operation of all of the individual switching circuits 184. In an embodiment, the controller 188 is a workstation provided in a control room for one or more system operators to review the performance of the system 180. In embodiments, the switching circuits 184 are controlled to fire in a predetermined sequence such that in each of a series of activation periods, fan shaped beams of X rays from one or more active source points propagate through an animal 185 passing on a ramp 187 through a center of the arrangement of X-ray tubes 181. Thus, in embodiments, the controller 188 is configured to control an activation and deactivation of each of the source points within each of the first and second linear multi-focus X-ray sources 145a, 145b.

It should be appreciated that, in various embodiments, the controller 188 implements a plurality of instructions or programmatic code to a) ensure that the switching circuits 184 are controlled to fire in a predetermined sequence, and b) perform process steps corresponding to various workflows and methods described in this specification.

Referring to FIGS. 1A and 1B, during a scanning operation, as the animal passes through the scanning region, area or aperture 150, each X-ray source point within an individual multi-focus X-ray source (145a, 145b) is switched on in turn and projection data through the animal as it passes is collected for that one source point. When the exposure is complete, a different X-ray source point is switched on, say, for example, within a different multi-focus X-ray source in the system 100 to create a next X-ray projection. The scanning process continues until all X-ray sources have been fired in a sequence that is configured to optimize a reconstructed X-ray image quality. In some embodiments, it is preferable to activate a non-adjacent source in the next part of the scanning sequence. In fact, it is preferable to activate a source at approximately 20 to 90 degrees away from a currently active source point. Thus, individual X-ray source points within the linear multi-focus X-ray sources 145a, 145b within each plane 142, 143 are active sequentially such that typically at least one X-ray beam is active at all times. In some embodiments, each source point within a first linear multi-focus X-ray source is switched on and subsequently, after going through each of the source points within the first linear multi-focus X-ray source, each source point within a second linear multi-focus X-ray source is switched on. In some embodiments, one source point within a first linear multi-focus X-ray source is switched on and then one source point within a second linear multi-focus X-ray source is switched on, thus, alternating back and forth (between the first and second linear multi-focus X-ray sources) until all source points have been activated.

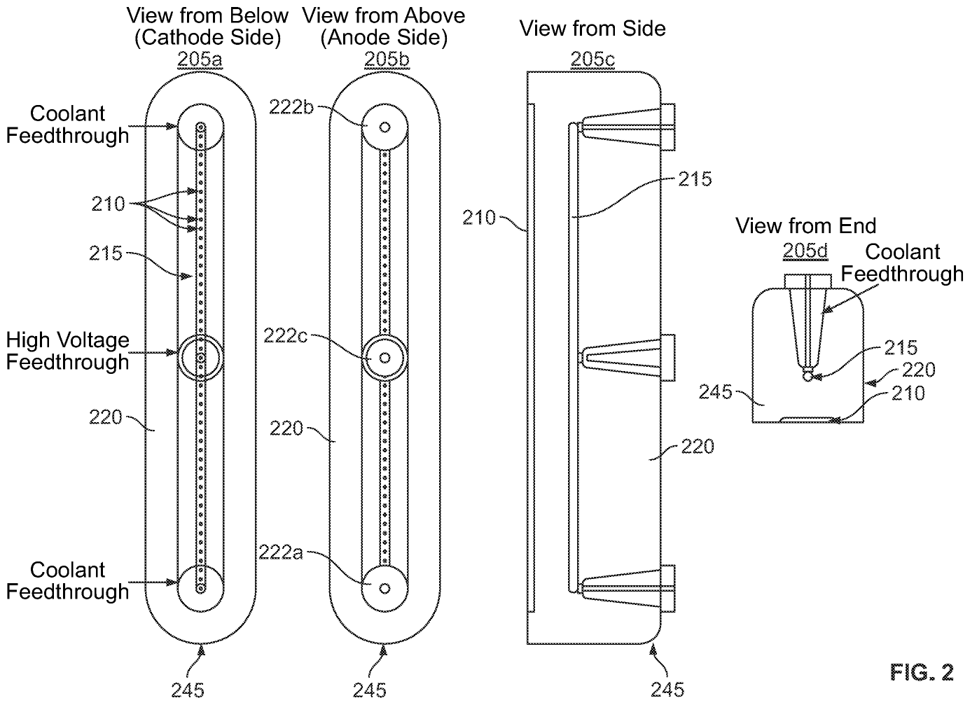

FIG. 2 shows bottom, top, longitudinal side and end views 205a, 205b, 205c, 205d of a linear multi-focus X-ray source 245 for use in a 3D stationary gantry X-ray CT imaging system, in accordance with some embodiments of the present specification. Referring now to the views 205a, 205b, 205c, 205d, simultaneously, the source 245 comprises a plurality of electron guns, cathodes or source/emission points 210 and an anode 215 housed in a vacuum tube or envelope 220. In some embodiments, the source 245 comprises 100 X-ray emission points 210 on 10 mm spacing over an active anode 215 of length 1000 mm.

In some embodiments, first, second and third supports 222a, 222b, 222c are deployed to support the anode 215 along a longitudinal axis. The first and second supports 222a, 222b are deployed at two ends while the third support 222c is deployed at the center of the anode 215. In some embodiments, the first and second supports 222a, 222b also function as coolant feed-through units while the third support 222c enables high voltage feed-through. In some embodiments, the anode 215 supports an operating tube voltage in a range of 100 kV to 300 kV. In some embodiments, each electron gun, cathode or source/emission point 210 emits a tube current in a range of 1 mA to 500 mA depending on animal thickness and inspection area, aperture or size—larger the inspection aperture and thicker the animal, higher the required tube current.

For scanning livestock (for example, cows and buffaloes), a suitable optimization is 225 kV tube voltage and 20 mA beam current, with total X-ray beam power of 4.5 kW. Coupled with tube filtration of minimum 3 mm aluminum this results in dose to the animal in a range of 2 μSv (microSievert) to 20 μSv, and in embodiments, around 10 μSv. To put this in context, typical individual dose to humans due to naturally occurring background radiation is 2 mSv/year (millisievert/year). An exposure of 10 μSv corresponds to 0.5% of one year of natural background radiation or around 2 days of natural background radiation.

In some embodiments, each electron gun 210 is configured to irradiate an area or focal spot on the anode 215 ranging between 0.5 mm to 3.0 mm diameters. Specific dimensions of the focal spot are selected to maximize image quality and minimize heating of the anode 215 during X-ray exposure. Higher the product of tube current and tube voltage, larger the focal spot is typically designed to be.

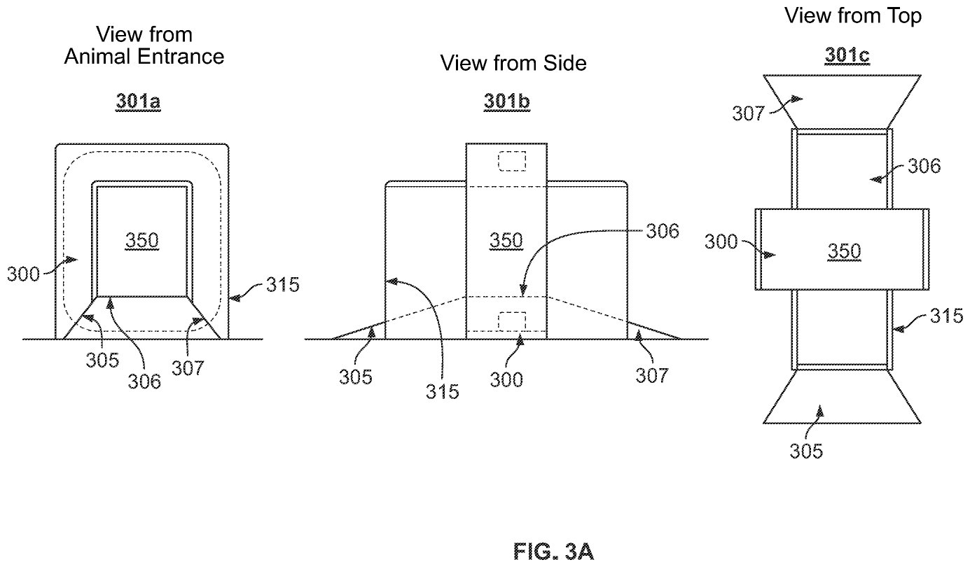

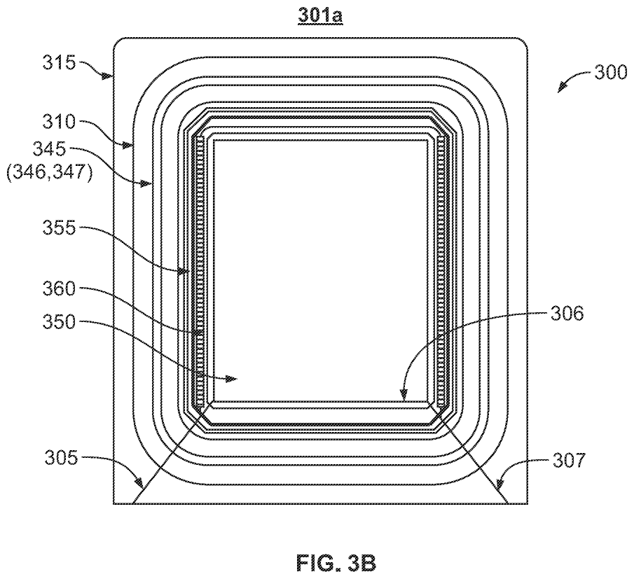

FIG. 3A illustrates first side, second side and top views 301a, 301b, 301c of a single-plane stationary gantry X-ray computed tomography system 300 configured to scan sheep, pigs and goats while FIG. 3B also illustrates the first side 301a, in accordance with some embodiments of the present specification. Referring to FIGS. 3A and 3B, the system 300 is deployed, for example, in an animal farm to scan livestock in real time as an animal passes through a scanning region, area, aperture or tunnel 350 of the system 300. The scanning region, area, aperture or tunnel 350 is smaller (compared to the scanning system of FIG. 1A, 1B) for scanning animals such as sheep, pigs and goats. The first side view 301a (FIGS. 3A, 3B) is in a direction parallel to the direction of motion of an animal as it passes through the scanning region, area or aperture 350 whereas the second side view 301b is in a direction perpendicular to the direction of motion of the animal passing through the scanning region, area or aperture 350.

In some embodiments, a first inclined ramp 305 is adapted to enable the animal to pass onto a horizontal platform 306 that lies in the scanning region, area or aperture 350 and eventually pass down using a second inclined ramp 307. In other words, the animal enters the scanning region, area or aperture 350 from the left in the view 301b and exits the scanning region, area or aperture 350 at the right in the 301b.

In some embodiments, the system 300 is enclosed within a food safe, environmentally protected enclosure 315 manufactured using materials such as, but not limited to, stainless steel, aluminum and/or plastic. In some embodiments, the system 100 is surrounded with at least one radiation shielding enclosure. In some embodiments, the system 300 has a multi-focus X-ray source 345 disposed in a plane around the scanning region, area or aperture 350. The source 345 comprises a plurality of X-ray source emission points, electron guns or cathodes 346 (also referred to as an electron gun array) around an anode 347. The plurality of X-ray source emission pints 346 and the anode 347 are enclosed in a vacuum envelope or tube 310. In some embodiments, the source 345 comprises 200 to 500 X-ray source emission points 346 arranged around a single anode 347 that is held at positive high voltage with respect to the corresponding electron gun array 346. In some embodiments, tube voltage is maintained in a range of 120 kV to 200 kV with tube current in a range 1 mA to 20 mA. In an embodiment, a single source 345 comprises a plurality of X-ray source emission points is employed for scanning small animals (such as, for example, sheep, pigs, and goats); while a plurality of linear multi-focus X-ray sources disposed around a scanning tunnel (such as, for example, shown in FIGS. 1A, 1B) are employed for scanning larger animals such as cattle. A preferred operating point for scanning small animals (such as, for example, sheep, pigs, and goats) is 160 kV, 4 mA corresponding to total X-ray beam power of 640 W. In embodiments, this results in a dose per scan to the animal on the order of 2 μSv to 20 μSv. In embodiments, the dose scan per animal is on the order of 10 μSv due to the smaller size of the scanning region, area, aperture or tunnel 350 (compared to the scanning region 150 of FIGS. 1A, 1B for beef scanning).

An array of detectors 355 is also positioned or deployed around the scanning region, area or aperture 350 to scan the animal as it passes through the scanning region, area or aperture 350. In some embodiments, the scanning region, area or aperture 350 has a substantially rectangular geometry or shape. In some embodiments, the scanning region, area or aperture 350 has a substantially square or polygonal geometry or shape. In some embodiments, the scanning region, area or aperture 350 has a width ranging from 400 mm to 800 mm and a height ranging from 600 mm to 1000 mm height. In an embodiment, as shown in FIG. 3, the scanning region 350 has a width of 600 mm and a height of 800 mm. In some embodiments, the array of detectors 355 are offset or displaced from the X-ray source 345 by a predefined distance so that X-rays from a source pass above the detector array adjacent to the source but interact in the detector array opposite to the source at the other side of the scanning region, area or aperture 350. In various embodiments, the predefined distance ranges from 2 mm to 20 mm.

A control room may be provided for one or more system operators to review the performance of the system 300 on one or more inspection workstations in data communication with the system 300. Alternatively, mobile computing devices may be used to inspect image data and control system operation. In various embodiments, the one or more inspection workstations are computing devices. At least one controller, positioned within the one or more inspection workstations, is configured to control an activation and deactivation of each of the plurality of X-ray source emission points.

It should be appreciated that, in various embodiments, the controller implements a plurality of instructions or programmatic code to a) ensure that the plurality of X-ray source emission points are controlled to fire in a predetermined sequence, and b) perform process steps corresponding to various workflows and methods described in this specification.

During a scanning operation, each X-ray source point within a multi-focus X-ray source is switched on, in turn, and where at least a portion of the X-rays pass through the animal, and the resultant projection data is collected for that one source point. When the exposure is complete, a different X-ray source point is switched on, for example, within a different multi-focus X-ray source (in embodiments that employ a plurality of linear multi-focus X-ray sources) to create a next X-ray projection. The scanning process continues until all X-ray sources have been fired/activated in a sequence that is configured to optimize a reconstructed X-ray image quality. In some embodiments, it is preferable to activate a non-adjacent source in the next part of the scanning sequence. In embodiments, it is preferable to activate a source positioned at approximately 20 to 90 degrees away from a currently active source point.

In embodiments employing a plurality of linear multi-focus X-ray sources, each source point within a first linear multi-focus X-ray source is switched on and then (only after going through each of the source points within the first linear multi-focus X-ray source) each source point within a second linear multi-focus X-ray source is switched on. In some embodiments employing a plurality of linear multi-focus X-ray sources, one source point within a first linear multi-focus X-ray source is switched on and subsequently, one source point within a second linear multi-focus X-ray source is switched on, thus, alternating back and forth (between the first and second linear multi-focus X-ray sources) until all source points have been activated.

In an embodiment, the system 300 comprises a series of X-ray source tubes operating in tandem, instead of the multi-focus X-ray source 345. In other words, the X-ray sources are a plurality of X-ray tubes and do not contain multiple source points.

While passing through the scanning region, area or aperture 350 the animal may move at an uncontrolled speed, especially if walking and not ambulatory, and may also possibly move from side to side. Consequently, the X-ray projection data needs to be motion corrected prior to implementing or executing back-projection algorithm. In some embodiments, this is enabled directly from the X-ray projection data itself by analyzing each set of data and forward projecting through the partial reconstructed X-ray data to see where the new projection is most likely to have come from. However, this is computationally expensive and so, in some embodiments, it is advantageous to use a secondary sensor system for monitoring surface profile of the animal and so measure motion directly. This information can then be used to determine where each new X-ray projection should be back-projected into the 3D reconstructed image volume.

Various types of 3D (three-dimensional) surface sensing technology may be used including, for example, point cloud optical and radar imaging sensors. FIG. 3B shows a radar imaging or inspection system 360 comprising radar transceivers or transceiver modules each comprising a plurality of Receiver (Rx) elements and Transmitter (Tx) elements which operate together to form a tomographic image of the animal as it passes through the scanning region, area or aperture 350.

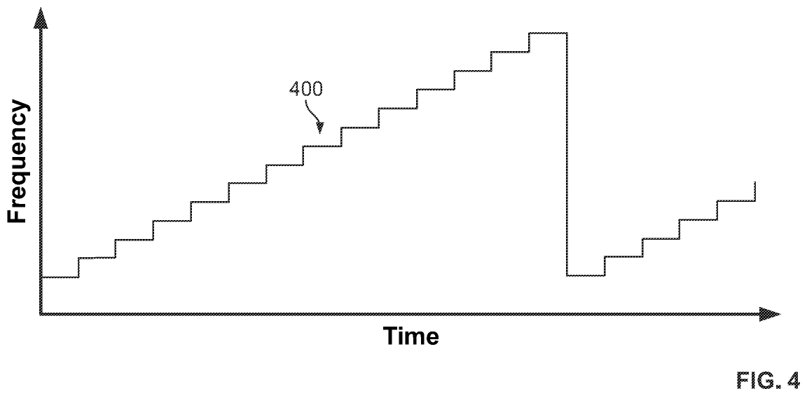

In some embodiments, the radar imaging or inspection system 360 is operated in stepped frequency continuous wave radar scanning sequence or mode 400, as shown in FIG. 4. In the radar scanning sequence or mode 400, each Tx element is held at a discrete set of frequencies (in the range 5 GHz to 50 GHz with 10 to 500 steps depending on range resolution required, in some embodiments) for a fixed period of time per step (1 to 100 μs, in some embodiments) in order to give time for the Rx elements to calculate phase and amplitude with respect to the input signal. Each Tx element is activated individually, with all of the Rx elements employed, listening in parallel to the radar signal from the individual Tx element, in order to form a tomographic data set which is then reconstructed to form a full surface image of the traversing animal. In one embodiment, for example, with Rx and Tx elements on 15 mm spacing over a 800 mm length, there are 50 Rx and Tx transceiver elements on each of first and second sides 350a, 350b of the scanning region, area or aperture 350. Assuming a 50 Hz (20 ms) imaging frame rate, for example, each Tx element will be active for 400 μs per ramp period. With an output frequency range from 10 GHz to 40 GHz and steps of 0.5 GHz (60 steps in total), the dwell time at each step is 6.5 μs, for example. In embodiments, the choice of which Tx element to activate at any time is made based upon the goal of maximizing the reconstructed image quality.

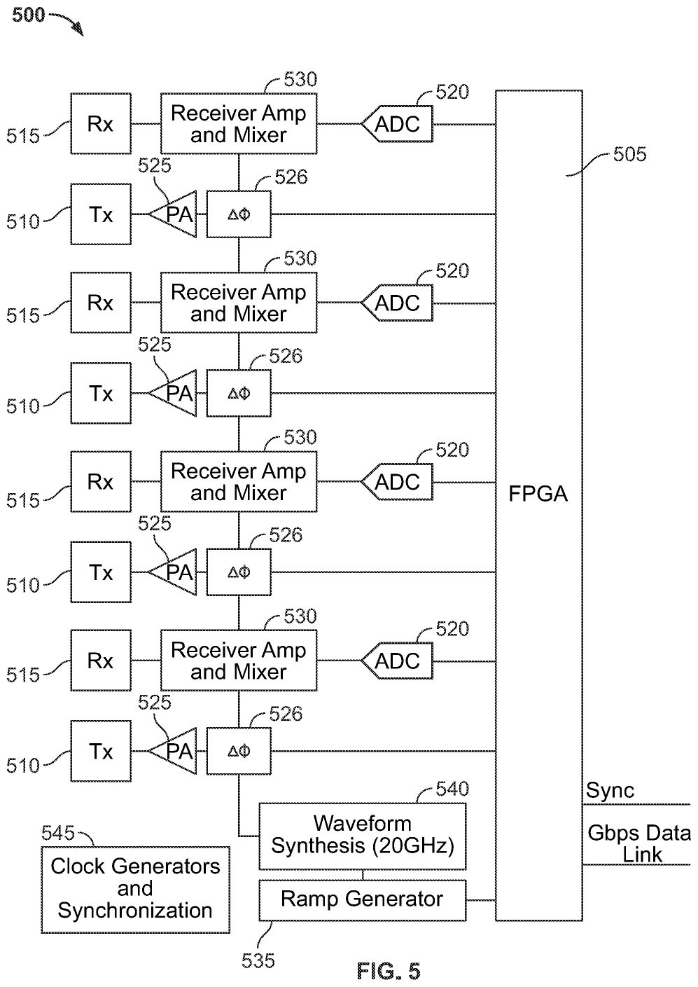

FIG. 5 is a block diagram of a radar imaging system 500 for determining body shape and body movement for correction of motion of an animal through an X-ray computed tomography scanning system, in accordance with some embodiments of the present specification. In some embodiments, the system 500 is an ultra-wide band radar system. In some embodiments, the system 500 comprises a field programmable gate array (FPGA) 505 to generate a base stepped frequency continuous wave signal which is frequency multiplied and amplified (at power amplifier elements 525) to each of a plurality of Tx transceiver elements 510 in turn. The FPGA 505 is coupled with the power amplifier elements 525 via frequency multiplier blocks 526, which convert low frequency CLOCK of the FPGA 505 (e.g. 100 MHz) to a higher output frequency CLOCK (e.g. 50 GHz) for the power amplifier elements 525. A ramp generator circuit 535, in communication with the FPGA 505, creates a linear rising or falling output signal with respect to time thereby producing a saw tooth waveform. A waveform synthesis element 540 receives the linear rising or falling waveform signal output from the ramp generator circuit 535 to output the base stepped frequency continuous wave signal for application to each of the plurality of Tx transceiver elements 510. A clock generator and synchronization circuit 545 produces a timing signal for use in synchronizing operation of the system 500.

In parallel, outputs from all Rx transceiver elements 515 are mixed with the Tx frequency, at Rx amplifier and mixer elements 530, to generate a lower frequency signal that can be measured by an analogue-to-digital converter (ADC) 520 and transferred to internal memories of the FPGA 505. Further, signal processing may be done in the FPGA 505 to reduce data bandwidth, or alternatively all data can be transferred through a high-speed interface to a host-computing device for processing.

In some embodiments, Tx and Rx transceiver elements 510, 515 employ circular polarization such that reflected waves return in an opposite polarization to the transmitted wave. This reduces cross talk between Tx and Rx transceiver elements 510, 515 thereby simplifying analogue front-end design as well as algorithmic complexity in image reconstruction.

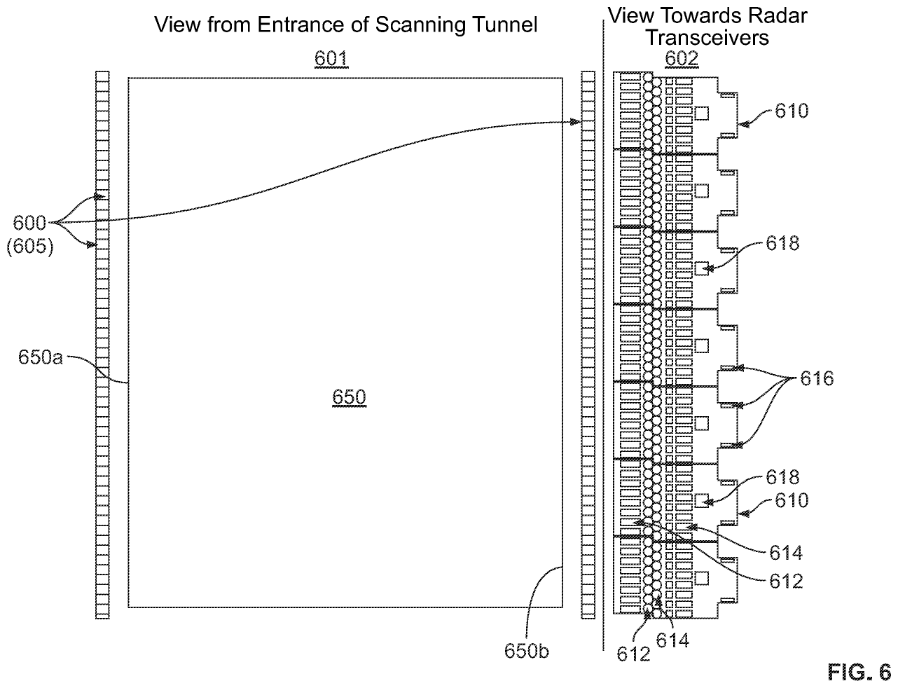

FIG. 6 shows an exemplary arrangement of a plurality of transmitter (Tx) and receiver (Rx) elements of a radar imaging or inspection system 600 deployed to determine body shape and movement for correction of motion of an animal through an X-ray computed tomography scanning system, in accordance with some embodiments of the present specification. The figure shows a view 601 along a direction parallel to a direction of motion of an animal through scanning region, area or aperture 650. The radar imaging or inspection system 600 comprises arrays 605 of radar Tx and Rx elements disposed on first and second vertical sides 650a, 650b of the scanning region, area or aperture 650.

Another view 602, along a direction perpendicular to the direction of motion of the animal through scanning region, area or aperture 650, shows a plurality of radar transceivers or transceiver modules 610, which may also be referred to as “cards” in some embodiments. Each of the transceivers 610 comprises a plurality of Tx and Rx elements (or analogue circuits) 612, 614. In some embodiments, each of the transceivers 610 comprises 8 Rx and 8. Tx elements 612, 614. In some embodiments, the Rx elements 614 are offset, in a vertical direction, by spacing of half an element from the Tx elements 612.

In some embodiments, the transmitter and receiver elements or analogue circuits 612, 614 with ADCs (Analog to Digital Circuits) are soldered to a same PCB (Printed Circuit Board) as the antenna structures with an overall FPGA for system control and data acquisition. Each of the transceivers 610 further comprises data transmission connectors 616 and a readout control circuit 618. Ribbon cables are used to transfer signals from one card to the next to allow flexibility in overall system configuration.

In accordance with some embodiments, each of the 3D X-ray computed tomography scanning systems of the present specification may be housed in a container that is located on the farm. When in use, doors at entry and exit ends of the container may be opened, the X-ray system powered up and scanning conducted by herding animals from the entry side of the container to the exit side of the container. In some embodiments, by reconciling RFID (Radio Frequency Identification) tag or other animal-specific IDs to the X-ray image data, quantitative information from an X-ray scan is associated back to individual animals to aid overall farm processes as well as food supply chain integrity process. In embodiments, containerized 3D X-ray computed tomography scanning systems may be installed permanently at the farm, or a particular container may be transported using a truck or trailer from one location to another as required to service multiple farms.

In accordance with some embodiments, the 3D X-ray computed tomography scanning systems of the present specification may be supported on mobile, roadworthy, scanning platforms such as, for example, a truck, van and/or a trailer. This enables the system to be transported on public and private roads to a required farm scanning site, the necessary scans conducted and the system then driven off to another farm where the scanning process can be repeated.

It should be noted that, in alternate embodiments, 3D high-resolution imaging methods such as, for example, magnetic resonance imaging, may be substituted for X-ray computed tomography. In addition, in various alternate embodiments, rotating gantry and/or single, dual and multi-plane stationary gantry X-ray computed tomography methods may be used interchangeably.

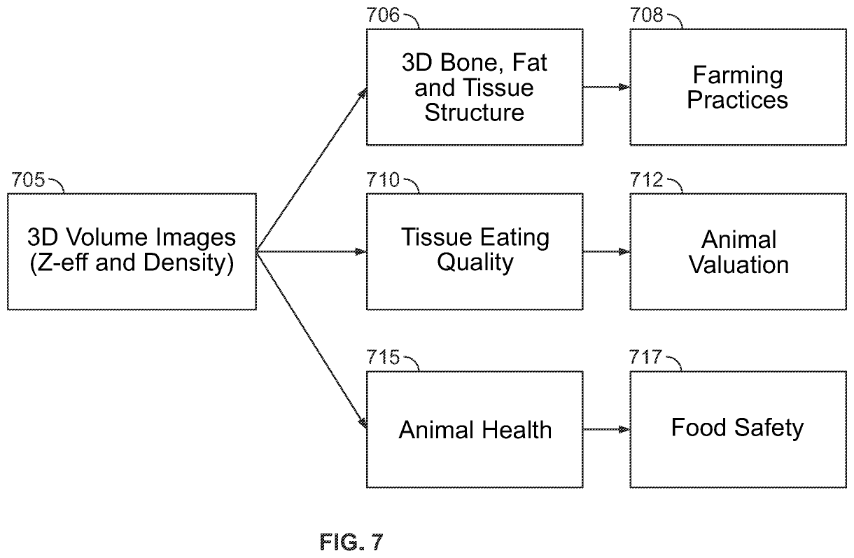

FIG. 7 is a block diagram of a plurality of exemplary information, outputs or outcomes derived based on processing or analyses of an animal's scan image data generated using a 3D stationary gantry X-ray CT imaging system, in accordance with some embodiments of the present specification. In embodiments, a controller in data communication with the 3D stationary gantry X-ray CT imaging system implements a plurality of instructions or programmatic code to receive 3D scan image data, process or analyze the scan image data and generate various outputs or outcomes such as, for example, effective Z, density information, 3D structure of the animal, calculating lean meat yield, analysis of intra-muscular fat, amount inter-muscular fat, ratio of intra-muscular fat (marbling) to tissue, absolute and relative size of individual organs, muscle volume, number of ribs, and presence or absence of cysts, tumors, pleurisy and foreign objects.

In accordance with aspects of the present specification, 3D scan image data of an animal provides effective Z (atomic number) and density information (block 705) leading to insight related to a 3D structure (comprising bony structure, size of each muscle and location and amount of fat) of the animal (block 706). This enables a farmer to optimize a plurality of farming processes (block 708) such as, for example, calculating lean meat yield and thereby determining how best to optimize a go forward plan for the herd including how much exercise, feed, feed supplements and water to include in the plan for the animal.

In some embodiments, the 3D scan image data of the animal is analyzed to deliver objective metrics or measurement data for all muscle groups within the animal, on an individual basis, in order to determine eating quality (block 710). In embodiments, the metrics or measurement data are determined by analysis of intra-muscular fat (marbling) and inter-muscular fat. As is known, inter-muscular fat is the fat that surrounds a muscle, and typically lies between the muscle and the skin of an animal. In embodiments, the metrics or measurement data are determined by analysis of a ratio of intra-muscular fat (marbling) to tissue. The farmer can use this data to plan to increase overall eating quality and/or to improve the quality of selected muscle groups in the highest value part of the animal—thereby leading to improved sale price or valuation of the animal (block 712).

In some embodiments, further analysis of the 3D image data provides metrics, measurement data or information on animal health (block 715) such as, for example, the absolute and relative size of individual organs (such as kidneys, liver, heart and lungs), the presence or absence of cysts and tumors, the presence of chronic conditions such as pleurisy and the presence of foreign objects such as barbed wire and needles that may lead to infection. Collectively information on animal health leads to improving overall quality control in food safety (block 717).

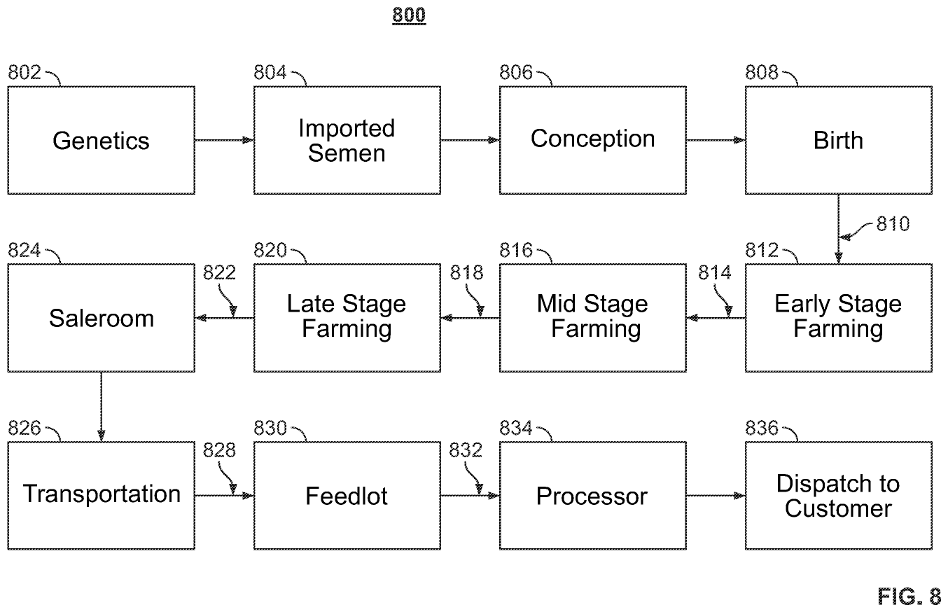

FIG. 8 is a workflow 800 illustrating use of a plurality of 3D X-ray computed tomography scanning processes during various events relating to farming of livestock, in accordance with some embodiments of the present specification. In accordance with aspects of the present specification, the workflow 800 illustrates an entire life cycle of an animal and a herd overall, from genetic selection through to the customer delivery, and a plurality of scanning points in the life cycle where 3D X-ray computed tomography at a farm may be beneficial. In embodiments, a controller in data communication with a 3D X-ray computed tomography scanner implements a plurality of instructions or programmatic code to implements a plurality of instructions or programmatic code to receive 3D scan image data, process or analyze the scan image data and generate various outputs or outcomes.

Blocks 802, 804, 806 and 808 respectively represent functions/events related to genetic selection, importing semen, conception and birth of an animal for rearing at the farm. At step 810, an initial/early scan of the animal is taken soon after birth, such as within 0-36 hours after birth, and in some cases longer, or before the animal reaches an age of 6 months using a 3D X-ray computed tomography scanning system such as those described with reference to FIGS. 1A, 1B, 3A, 3B and 6. Data from this initial/early scan is directed towards identifying abnormalities and also for checking predefined genetic features. For example, most lambs are born with 8 ribs, but some are born with 7 ribs and some with 9 ribs. It is helpful to know how many ribs the lamb contains from an early stage since an ultimate value of the animal may be dependent on such information. The identified abnormalities and predefined genetic features are recorded in at least one database.

In embodiments, 3D X-ray computed tomography scans are taken of an animal during various stages of development. For example, in embodiments, at a first stage of development, an animal may be in a first age range, beginning at a first start date and ending at a first end date. In embodiments, a first stage of development corresponds to an early stage. In embodiments, at a second stage of development, an animal may be in a second age range beginning at a second start date and ending at a second end date. In embodiments, a second stage of development corresponds to a mid-range stage. In embodiments, at a third stage of development, an animal may be in a third age range beginning at a third start date and ending at a third end date. In embodiments, the third stage of development corresponds to a late stage. In embodiments, at a fourth stage of development, an animal may be in a fourth age range beginning at a fourth start date and ending at a fourth end date.

In embodiments, the first start date corresponds to the date of birth of the animal and is before each of the first end date, the second start date, the second end date, the third start date, the third end date, the fourth start date, and the fourth end date.

In embodiments, the first end date is after each of the first start date and before each of the second start date, the second end date, the third start date, the third end date, the fourth start date, and the fourth end date.

In embodiments, the second start date is after each of the first start date and the first end date and before each of the second end date, the third start date, the third end date, the fourth start date, and the fourth end date.

In embodiments, the second end date is after each of the first start date, the first end date and the second start date and before each of the third start date, the third end date, the fourth start date, and the fourth end date.

In embodiments, the third start date is after each of the first start date, the first end date, the second start date, and the second end date and before each of the third end date, the fourth start date, and the fourth end date.

In embodiments, the third end date is after each of the first start date, the first end date, the second start date, the second end date, and the third start date and before each of the fourth start date and the fourth end date.

In embodiments, the fourth start date is after each of the first start date, the first end date, the second start date, the second end date, the third start date, and the third end date and before each of the fourth end date.

In embodiments, the fourth end date is after each of the first start date, the first end date, the second start date, the second end date, the third start date, the third end date, and the fourth start date.

In embodiments, there may be n stages of development, with nth start dates and nth end dates, appearing in chronological order as described above. In embodiments, the first end date may be on the same day as, or one day, before the second start date. In embodiments, the second end date may be on the same day as, or one day before, the third start date. In embodiments, the third end date may be on the same day as, or one day before, the fourth start date. In embodiments, the fourth end date may be the same day as, or one day before, the nth start date. It should be noted that the various age ranges of development is dependent upon the animal species.

At step 814, a 3D X-ray computed tomography scan of the animal is acquired after the animal completes a first stage (block 812) in development, that is, when the animal is in a first age range. The scan at step 814 is directed towards determining any abnormalities or health conditions (such as, for example, presence or absence of cysts, tumors, pleurisy and foreign objects) that may affect the ultimate value of the animal.

At step 818, another 3D X-ray computed tomography scan of the animal is acquired after the animal completes a mid-stage (block 816) in development, that is, when the animal is in a second age range. The quality control scan at step 818 enables driving optimization of the animal and the herd as a whole. It is at this stage that significant transformation in valuation can be achieved of the animal and the herd.

At step 822, a yet another scan of the animal is acquired once the animal has been reared through late-stage farming (block 820) and is ready to leave the farm, that is, when the animal is in a third age range. The 3D X-ray computed tomography scan, at step 822, is used to generate a complete analysis of the animal (to generate metrics or measurement data such as, for example, lean meat yield, localized eating quality and health) which together describe the animal sufficiently for presentation at auction and so achieve a final purchase price. In various embodiments, data from scan steps 818 and 822 is used to determine a value of the animal based on at least one of a plurality of pre-sale parameters including lean meat yield, ratio of intra-muscular fat to tissue, amount of inter-muscular fat, absolute and relative size of individual organs, muscle volume, number of ribs, and presence or absence of cysts, tumors, pleurisy and foreign objects.

It is known that transfer of animals from the farm to sale yards is stressful for the animal and expensive for the farmer. Therefore, the ability to conduct virtual auctions with electronic data, including that from the 3D X-ray computed tomography data, is beneficial.

Following sale and transportation (blocks 824, 826 respectively) of the animal from the farm, a 3D X-ray computed tomography scan is acquired at a feedlot, at step 828. The scan at step 828 is directed towards performing an incoming check of the animal post auction to validate the electronic data that was presented at auction and also to check on animal health where animals from multiple herds are being combined. Thus, data from the scan at step 828 is used to determine one or more of a plurality of after-sale parameters. In embodiments, the validation of the electronic data involves comparing at least a portion of the plurality of pre-sale parameters with at least a portion of a plurality of after-sale parameters. In embodiments, the plurality of after-sale parameters include lean meat yield, ratio of intra-muscular fat to tissue, amount of inter-muscular fat, absolute and relative size of individual organs, muscle volume, number of ribs, and presence or absence of cysts, tumors, pleurisy and foreign objects.

At step 832, a final scan of the animal is conducted at the end of the feedlot process (block 830) where the animal has generally been fattened prior to slaughter. This final scan provides initial data to enable planning production schedules/processes (block 834) and hence optimize a factory process and, thereafter, final dispatch to customers (block 836).

Persons of ordinary skill in the art should appreciate that, in some embodiments, the scan information generated on an animal at a particular stage in development is aggregated with information from other animals at similar and different stages in development to determine, using methods such as (for example) artificial intelligence and big data analytics, a predicted outcome for the animal as well as an impact on overall development of a herd within a particular farm and also between different farms.

In embodiments, multi-energy computed tomography and transmission X-ray screening may be employed for the purposes of the present specification. In embodiments, the use of multi-energy transmission X-ray screening enables improved Z_{eff} recovery in single-view and stereo-view imaging systems leading to improved chemical lean accuracy and improved location of bone structure especially in high attenuation regions. In addition, the use of multi-energy transmission X-ray screening enables improved Zeff recovery for use in foreign object detection and final product quality control.

In embodiments, the technologies described above may be integrated with meat processing and plant safety practices. In embodiments, the present specification employs the use of software to link three-dimensional imaging and multi-energy meat processing technology to plant operations. In embodiments, the present specification employs the use of software to link three-dimensional imaging and multi-energy meat processing technology to farming practices. In embodiments, the present specification employs the use of modified security technology, such as personnel and baggage screening systems, such that these technologies can be employed within the meat industry across several applications.

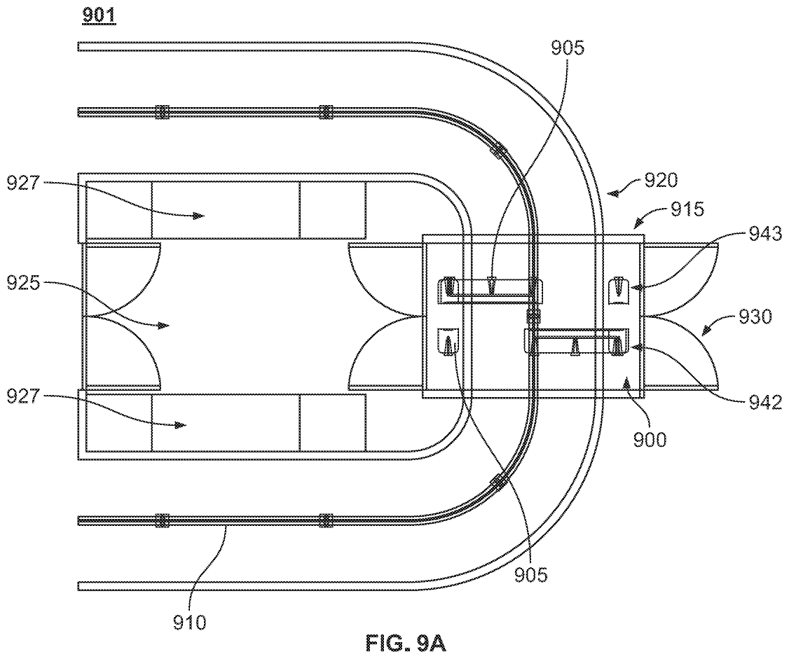

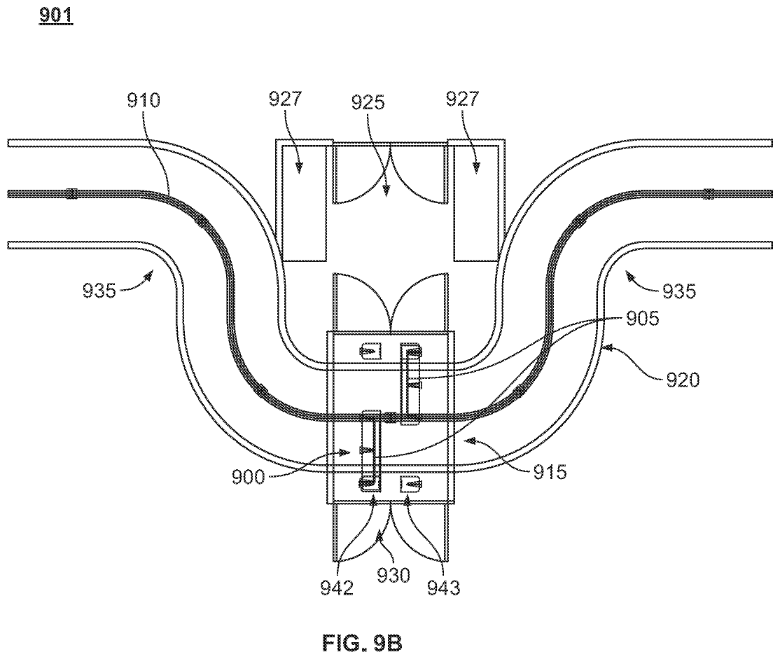

FIGS. 9A and 9B illustrate top views of a 3D stationary gantry X-ray CT imaging system 900 (also referred to as a Real-Time Tomography (RTT) system) in first and second configurations, respectively, to scan meat in an abattoir 901, in accordance with some embodiments of the present specification. Referring now to FIGS. 9A and 9B, the system 900 is deployed in the abattoir 901 to scan carcasses 905 hanging from hooks of a conveyor or conveyor rail 910 and being moved through the system 900 by the conveyor rail 910. In some embodiments, the carcasses 905 are moved, by the conveyor rail 910, through the system 900, at a speed ranging from 0.05 m/s to 0.5 m/s.

In some embodiments, the system 900 is enclosed within a food safe, environmentally protected enclosure 915 manufactured using materials such as, but not limited to, stainless steel and/or plastic. In some embodiments, the system 900 is surrounded with at least one radiation shielding enclosure or tunnel 920. A control room 925 is provided for one or more system operators to review the performance of the system 900 on one or more inspection workstations 927. A service access 930 is also provided to the system 900. In various embodiments, the one or more inspection workstations 927 are computing devices.

In some embodiments, the system 900 is configured for dual-plane scanning of carcasses and comprises a first plurality of linear multi-focus X-ray sources along with an associated first array of detectors positioned or deployed around an inspection region, area or aperture to scan carcasses in a first imaging plane 942 and a second plurality of linear multi-focus X-ray sources along with an associated second array of detectors also positioned or deployed around the inspection region, area or aperture to scan carcasses in a second imaging plane 943. In some embodiments, the first and second imaging planes 942, 943 are along a direction parallel to the direction of motion of the carcasses along the conveyor rail 910. In embodiments, the first plurality of linear multi-focus X-ray sources are offset from the associated first array of detectors, in the first imaging plane 942, by a distance d₁ while the second plurality of linear multi-focus X-ray sources are offset from the associated second array of detectors, in the second imaging plane 943, by a distance d₂. In some embodiments, d₁ is equal to d₂. In various embodiments, the distances d₁ and d₂ range from 1 mm to 10 mm.

In some embodiments, as shown in FIG. 9A, the at least one radiation shielding enclosure 920 as well as the conveyor rail 910 may have a layout similar to a labyrinth or maze such that there is no straight path through the at least one radiation shielding enclosure 920 and the conveyor rail 910 and that any path through the at least one radiation shielding enclosure 920 and the conveyor rail 910 requires at least more than 1 turn and less than 20 turns, preferably in the range of 2 to 5 turns, each turn having a turning radius greater than 10 percent. In addition, each turn is preferably in the range of 25 to 80 degrees and any increment therein. In embodiments, the labyrinthine layout serves to restrict radiation exposure to workers in the abattoir 901 to below statutory limits (typically, less than 1 uSv/hr in any one hour). In some embodiments, as shown in FIG. 9B, the at least one radiation shielding enclosure 920 as well as the conveyor rail 910 have a layout in a broadly linear fashion but with one or more deviations, chicanes, or turns 935 to restrict radiation exposure to workers in the abattoir 901 below statutory limits. Persons of ordinary skill in the art would appreciate that the configurations of FIGS. 9A and 9B are only exemplary and in no way limiting. For example, in alternate embodiments, the at least one radiation shielding enclosure 920 as well as the conveyor rail 910 may have other layout configurations such as, but not limited to, an elbow or a staircase.

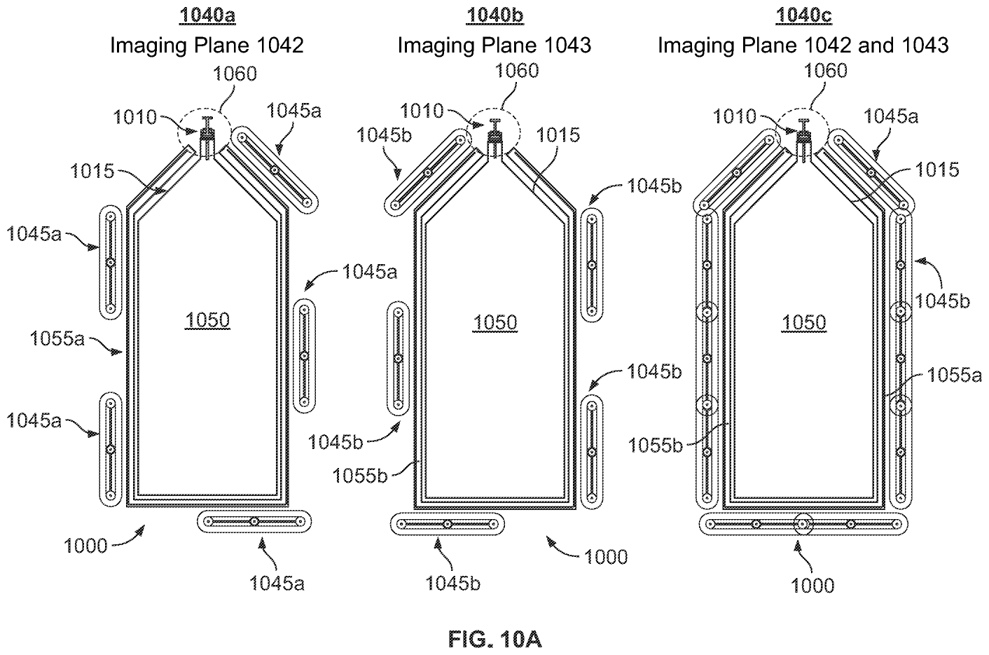

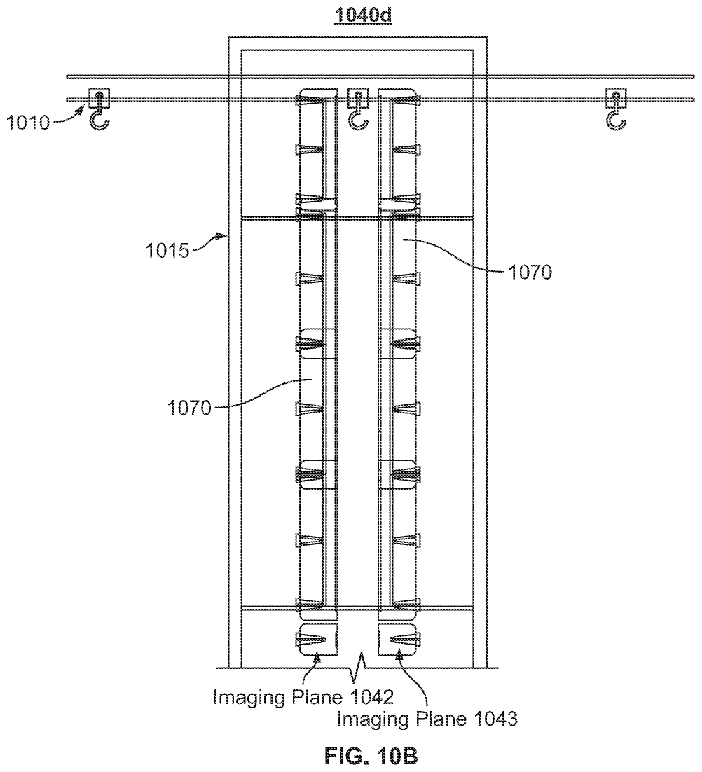

FIG. 10A illustrates first, second and third cross-sectional views 1040a, 1040b, 1040c of a 3D stationary gantry X-ray CT imaging system 1000 configured for dual-plane scanning of carcasses, in accordance with some embodiments of the present specification. The first cross-sectional view 1040a is along a direction parallel to the direction of motion of carcasses along a conveyor rail 1010 and perpendicular to a first imaging plane 1042. In embodiments, the first imaging plane 1042 comprises a plurality of separate linear multi-focus X-ray sources 1045a arranged around an inspection area 1050. In some embodiments, the first imaging plane 1042 comprises, say, five linear multi-focus X-ray sources 1045a separated from each other and positioned around or along a perimeter of the inspection area 1050.

The inspection area or aperture 1050 is bounded by a food safe environmental enclosure or housing 1015. The inspection area or aperture 1050 is surrounded by an array of X-ray detectors 1055a positioned in the first imaging plane 1042 such that the X-ray detectors 1055a lie between the linear multi-focus X-ray sources 1045a and the housing 1015. The array of detectors 1055a is offset, by a distance of 1 mm to 10 mm from the plane of the X-ray sources 1045a such that X-rays from a multi-focus X-ray source on one side of the inspection aperture 1050 can pass above the adjacent X-ray detectors and interact with X-ray detectors on an opposing side of the inspection area 1050, thereby forming a transmission image through a carcass under inspection.

The second cross-sectional view 1040b is along the direction parallel to the motion of carcasses along the conveyor rail 1010 and perpendicular to a second imaging plane. In embodiments, the second imaging plane also comprises a plurality of separate linear multi-focus X-ray sources 1045b arranged around the inspection area 1050. In some embodiments, the second imaging plane 1043 comprises, say, five linear multi-focus X-ray sources 1045b separated from each other and positioned around or along the perimeter of the inspection area 1050. In some embodiments, the five linear multi-focus X-ray sources 1045b (in the second imaging plane 1043) are disposed or positioned so as to fill the gaps separating the five linear multi-focus X-ray sources 1045a (in the first imaging plane 1042).

The inspection area or aperture 1050 is surrounded by another array of X-ray detectors 1055b positioned in the second imaging plane 1043 such that the X-ray detectors 1055b lie between the linear multi-focus X-ray sources 1045b and the housing 1015. The array of detectors 1055b is also offset, by a few millimeters, from the plane of the X-ray sources 1045b such that X-rays from a multi-focus X-ray source on one side of the inspection aperture 1050 can pass above the adjacent X-ray detectors and interact with X-ray detectors on an opposing side of the inspection area 1050, thereby forming a transmission image through the carcass under inspection.

The third cross-sectional view 1040c illustrates a composite representation of the first and second imaging planes 1042, 1043 as the carcass moves through the system 1000. The view 1040c shows a complete locus of multi-focus X-ray source points about the inspection area 1050 as required to form a high-quality 3D tomographic image of the carcass. A small region 1060 of missing data is observable adjacent to a hook on which the carcass is transported. Accordingly, an image reconstruction algorithm of the system 1000 is configured to minimize an impact of the missing data in a final image.

During a scanning operation, each X-ray source point within an individual multi-focus X-ray source (1045a, 1045b) is switched on in turn and projection data through the carcass is collected for that one source point. When the exposure is complete, a different X-ray source point is switched on, say, for example, within a different multi-focus X-ray source in the system 1000 to create a next X-ray projection. The scanning process continues until all X-ray sources have been fired in a sequence that is configured to optimize a reconstructed X-ray image quality.

In some embodiments, the inspection area 1050 has a cross-sectional shape, which is a composite of a first rectangular shape mounted by a second triangular shape. In some embodiments, the first rectangular cross-sectional shape has an exemplary size defined by a width that is less than 20%, preferably less than 40% of a height. In some embodiments, the first rectangular cross-sectional shape has an exemplary size (area) of 1500 mm (width)×3900 mm (height). In some embodiments, the area of the second triangular shape is substantially less or negligible compared to the area of the first rectangular shape. Therefore, for practical purposes, the exemplary size (area) of 1500 mm (width)×3900 mm (height) for the first rectangular cross-sectional shape is representative of the composite—that is, the inspection area 1050. It should be appreciated that this size (area) of 1500 mm (width)×3900 mm (height) of the inspection area or aperture 1050 is suited to scanning beef carcasses, in some embodiments.

FIG. 10B illustrates a fourth cross-sectional view 1040d of the 3D stationary gantry X-ray CT imaging system 1000, in accordance with some embodiments of the present specification. The fourth cross-sectional view 1040d is along a direction perpendicular to the direction of motion of carcasses 1070 along the conveyor rail 1010 and parallel to the first and second imaging planes 1042, 1043. In embodiments, the first and second imaging planes 1042, 1043 are separated by a distance thereby simplifying service access. In some embodiments, the distance ‘d’ ranges from 100 mm to 2000 mm. In an embodiment, the distance ranges from 500 mm to 1000 mm. In embodiments, the carcass motion that may occur between the first and second imaging planes 1042, 1043 other than that in a simple linear direction (for example, in a front to back and/or left to right swinging and/or rotational motion) may be measured using standard 3D optical point cloud or radar imaging methods known to persons of ordinary skill in the art. This 3D data may be converted to actual carcass displacement at each point in the field and used to drive the tomographic image reconstruction back-projection process. This may be achieved through methods known to those skilled in the art such as, for example, re-calculation of the direction of each X-ray projection from source to detector through a virtual carcass, in computer memory, as the image reconstruction process takes place.

According to aspects of the present specification, a size of an inspection region can be configured for specific carcass-based applications by deploying a specific imaging geometry comprising a) selecting the number and position of multi-focus X-ray sources (such as, sources 1045a, 1045b) to be used and b) configuring the array of X-ray detectors (such as, detectors 1055a, 1055b) to suit the X-ray source positions. The specific imaging system geometry is passed to the X-ray 3D image reconstruction algorithm where a one-time re-calculation of weighting functions is conducted to ensure accurate image reconstruction. The embodiments of FIGS. 9A, 9B, 10A and 10B are representative of the type of imaging system that might be deployed in an abattoir processing beef. A comparatively smaller inspection area or aperture is typically required in abattoirs processing pigs, goats and lamb. It should be appreciated that below an inspection area or aperture of approximately 1 m diameter, it is typically more cost effective to use a single-plane scanning system such as a rotating gantry computed tomography system or a stationary gantry imaging system with a rectangular or circular tube configuration.

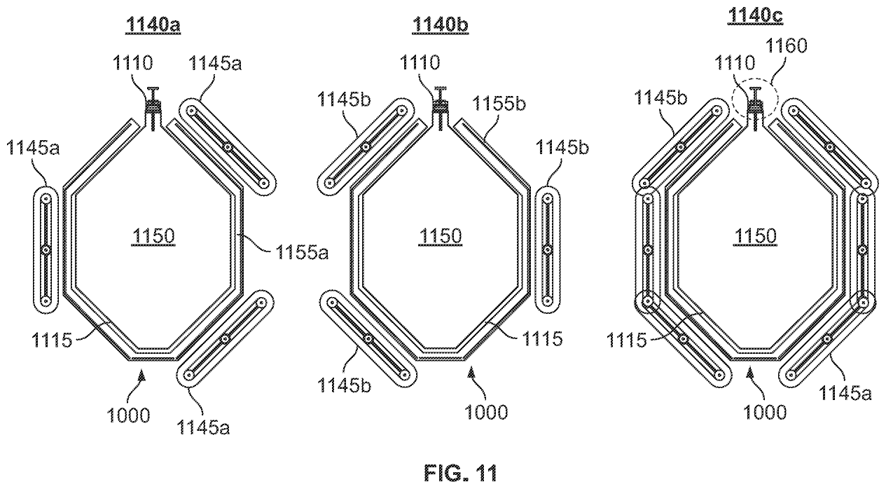

For example, FIG. 11 illustrates first, second and third cross-sectional views 1140a, 1140b, 1140c of a 3D stationary gantry X-ray CT imaging system 1100 configured for dual-plane scanning of carcasses, in accordance with some embodiments of the present specification. The first cross-sectional view 1140a is along a direction parallel to the motion of carcasses along a conveyor rail 1110 and perpendicular to a first imaging plane. In embodiments, the first imaging plane comprises a plurality of separate linear multi-focus X-ray sources 1145a arranged around an inspection region, area or aperture 1150. In some embodiments, the first imaging plane comprises, say, three linear multi-focus X-ray sources 1145a separated from each other and positioned around or along a perimeter of the inspection area 1150.

In accordance with an aspect of the present specification, the inspection area or aperture 1150 has a polygonal geometry or shape to approximate a round or circular cross-section. The polygonal shape or geometry is suited to scan carcasses of lamb, pigs and goats. In some embodiments, the inspection area or aperture 1150 has a maximum width of 1500 mm and a maximum height of 2000 mm. In some embodiments, the inspection area or aperture 1150 has a maximum width that is less than 10%, preferably less than 20% of a maximum height.

In some embodiments, the inspection area or aperture 1150 is bounded by a food safe environmental enclosure or housing 1115. The inspection area or aperture 1150 is surrounded by an array of X-ray detectors 1155a positioned in the first imaging plane such that the X-ray detectors 1155a lie between the linear multi-focus X-ray sources 1145a and the housing 1115. The array of detectors 1155a is offset, by a few millimeters, from the plane of the X-ray sources 1145a such that X-rays from a multi-focus X-ray source on one side of the inspection aperture 1150 can pass above the adjacent X-ray detectors and interact with X-ray detectors on an opposing side of the inspection area 1150, thereby forming a transmission image through a carcass under inspection.

The second cross-sectional view 1140b is along the direction parallel to the motion of carcasses along the conveyor rail 1110 and perpendicular to a second imaging plane. In embodiments, the second imaging plane also comprises a plurality of separate linear multi-focus X-ray sources 1145b arranged around the inspection area 1150. In some embodiments, the second imaging plane comprises, say, three linear multi-focus X-ray sources 1145b separated from each other and positioned along the perimeter of the inspection area 1150. In some embodiments, the three linear multi-focus X-ray sources 1145b (in the second imaging plane) are disposed or positioned so as to fill the gaps separating the three linear multi-focus X-ray sources 1145a (in the first imaging plane).

The inspection area or aperture 1150 is surrounded by another array of X-ray detectors 1155b positioned in the second imaging plane such that the X-ray detectors 1155b lie between the linear multi-focus X-ray sources 1145b and the housing 1115. The array of detectors 1155b is also offset, by a few millimeters, from the plane of the X-ray sources 1145b such that X-rays from a multi-focus X-ray source on one side of the inspection aperture 1150 can pass above the adjacent X-ray detectors and interact with X-ray detectors on an opposing side of the inspection area 1150, thereby forming a transmission image through the carcass under inspection.

The third cross-sectional view 1140c illustrates a composite representation of the first and second imaging planes as the carcass moves through the system 1100. The view 1140c shows a complete locus of multi-focus X-ray source points about the inspection area 1150 as required to form a high-quality 3D tomographic image of the carcass. A small region 1160 of missing data is observable adjacent to a hook on which the carcass is transported. Accordingly, an image reconstruction algorithm of the system 1100 is configured to minimize an impact of the missing data in a final image.

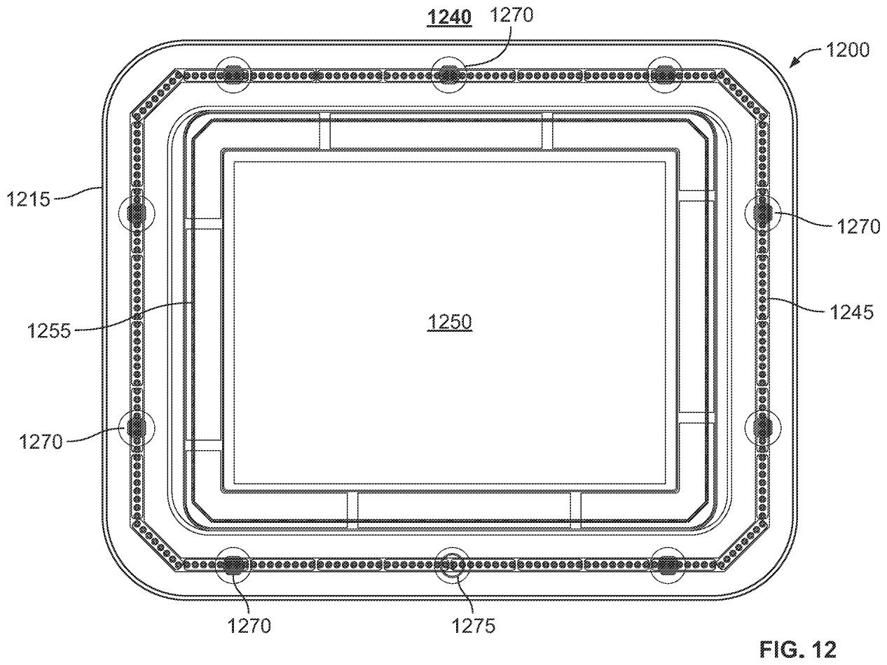

As another example, FIG. 12 illustrates a cross-sectional view 1240 of a 3D stationary gantry X-ray CT imaging system 1200 configured for single-plane scanning of carcasses, in accordance with some embodiments of the present specification. The system 1200 comprises a plurality of separate linear multi-focus X-ray sources 1245 arranged around a perimeter of an inspection region, area or aperture 1250. The inspection region, area or aperture 1250 is surrounded by an array of X-ray detectors 1255. The plurality of separate linear multi-focus X-ray sources 1245 and the array of X-ray detectors 1255 are enclosed in a housing 1215.

The figure also shows a plurality of first structures 1270 for enabling heat dissipation from the plurality of X-ray sources 1245 and at least one second structure 1275 for enabling heat dissipation from and also for providing voltage supply to the plurality of X-ray sources 1245. In embodiments, the first structure 1270 is designed to maximize mechanical integrity and heat conductivity. The at least one second structure 1275 comprises a thermally conductive element to dissipate heat from an anode region and also a metal rod that passes through its center to supply voltage.

In accordance with an aspect of the present specification, the inspection region, area or aperture 1250 has a substantially non-circular geometry or shape such as rectangular or square, for example. The rectangular or square shape or geometry is suited to scan whole poultry and beef, lamp, pig and goat carcass sections during de-boning process. In some embodiments, the inspection area or aperture 1250 has a size of 600 mm (width)×450 mm (height).

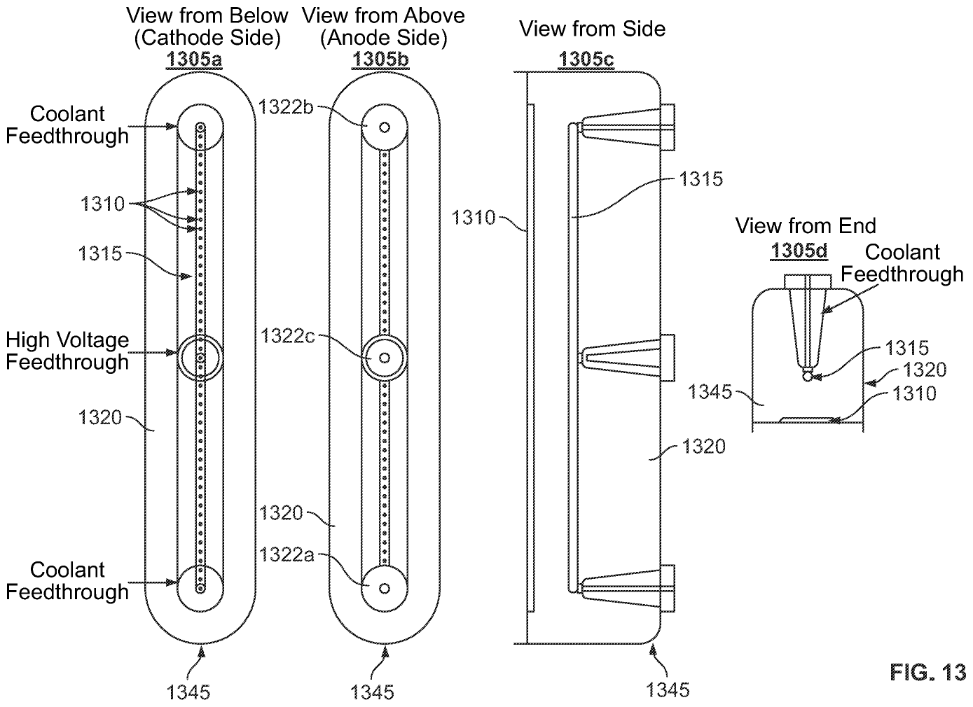

FIG. 13 shows bottom, top, longitudinal side and end views 1305a, 1305b, 1305c, 1305d of a linear multi-focus X-ray source 1345 for use in a 3D stationary gantry X-ray CT imaging system, in accordance with embodiments of the present specification. Referring now to the views 1305a, 1305b, 1305c, 1305d, simultaneously, the source 1345 comprises a plurality of electron guns, cathodes or source/emission points 1310 and an anode 1315 housed in a vacuum tube or envelope 1320. In some embodiments, the source 1345 comprises 100 X-ray emission points 1310 on 10 mm spacing over an active anode 1315 of length 1000 mm.

In some embodiments, first, second and third supports 1322a, 1322b, 1322c are deployed to support the anode 1315 along a longitudinal axis. The first and second supports 1322a, 1322b are deployed at two ends while the third support 1322c is deployed at the center of the anode 1315. In some embodiments, the first and second supports 1322a, 1322b also function as coolant feed-through units while the third support 1322c enables high voltage feed-through. In some embodiments, the anode 1315 supports an operating tube voltage in a range of 100 kV to 300 kV. In some embodiments, each electron gun, cathode or source/emission point 1310 emits a tube current in a range of 1 mA to 500 mA depending on carcass thickness and inspection area, aperture or size—larger the inspection aperture and thicker the carcass, higher the required tube current.

In some embodiments, each electron gun 1310 is configured to irradiate an area or focal spot on the anode 1315 ranging between 0.5 mm to 3.0 mm diameters. Specific dimensions of the focal spot are selected to maximize image quality and minimize heating of the anode 1315 during X-ray exposure. Higher the product of tube current and tube voltage, larger the focal spot is typically designed to be.

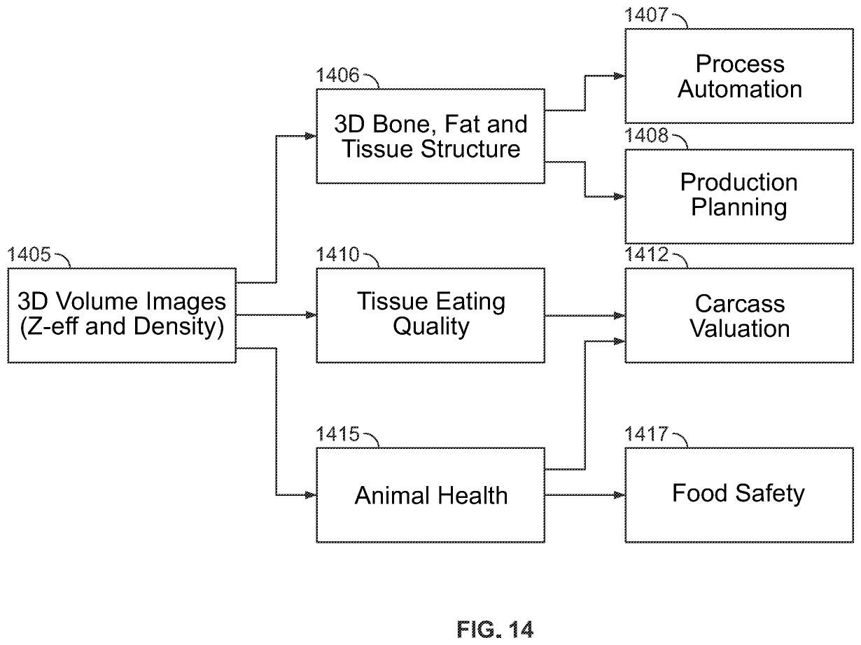

FIG. 14 is a block diagram illustration of a plurality of exemplary information, outputs or outcomes derived based on processing of carcass scan image data generated using a dual-plane 3D stationary gantry X-ray CT imaging system, in accordance with some embodiments of the present specification. In embodiments, a controller in data communication with the 3D stationary gantry X-ray CT imaging system implements a plurality of instructions or programmatic code to receive 3D scan image data, process or analyze the scan image data and generate various outputs or outcomes such as, for example, effective Z, density information, 3D structure of the animal, calculating lean meat yield, analysis of intra-muscular fat, amount inter-muscular fat, ratio of intra-muscular fat (marbling) to tissue, absolute and relative size of individual organs, muscle volume, number of ribs, and presence or absence of cysts, tumors, pleurisy and foreign objects.

In accordance with aspects of the present specification, 3D scan image data of a carcass provides effective Z (atomic number) and density information (block 1405) leading to insight related to the 3D structure (comprising bone, fat and tissue structure) of the carcass (block 1406) and therefore may be used to drive a system for automatic cutting (block 1407) of the carcass based on its structure. It is known, for example, that lamb carcasses have 8 ribs typically, but sometimes a lamb may have just 7 or even 9. To continue this example, in order to plan optimal output from an abattoir, it needs to be determined as to how many lamb chops are required as opposed to rack of lamb, which typically comprises 7 ribs. Therefore, a carcass may yield 1 rack, 1 rack and 1 chop or 1 rack and 2 chops. The decision on whether the carcass should be processed into individual chops or into rack plus chop(s) is ideally made prior to the start of a day's production. In some embodiments, therefore, the use of 3D imaging can drive optimal production planning (block 1408) and establish a correct cutting sequence for one or more automated cutting equipment.

In some embodiments, the 3D scan image data of the carcass can also be used to determine eating quality (block 1410) in 3D within the carcass as a whole. It is known that the density of fat and muscle are dissimilar. Therefore, they appear at different grey levels in the reconstructed X-ray image. Metrics of eating quality in beef, for example, are determined by a) a ratio of intra-muscular fat to tissue (marbling) as well as b) an amount of inter-muscular fat. Analysis of eating quality through these metrics, at each point in each muscle, determines a first amount or portion of each muscle within the carcass that will be destined for highest value output, a second amount or portion that will be destined for standard output and a third amount or portion that will be destined for low value output. This analysis drives the overall valuation (block 1412) of the carcass and ensures that farmers can be remunerated fairly for producing high quality animals and not simply on carcass weight or lean meat yield (the percentage of meat, fat and bone in the carcass).

In some embodiments, further analysis of the 3D image data provides information on carcass/animal health (block 1415), for example the presence of foreign objects such as syringe needles and barbed wire inclusions, and also the presence of cysts and tumors, oversized organs, pleurisy and other common diseases. Collectively, this information also drives carcass valuation since an unhealthy carcass will be diverted to a low value food chain while simultaneously improving overall quality control in food safety (block 1417).

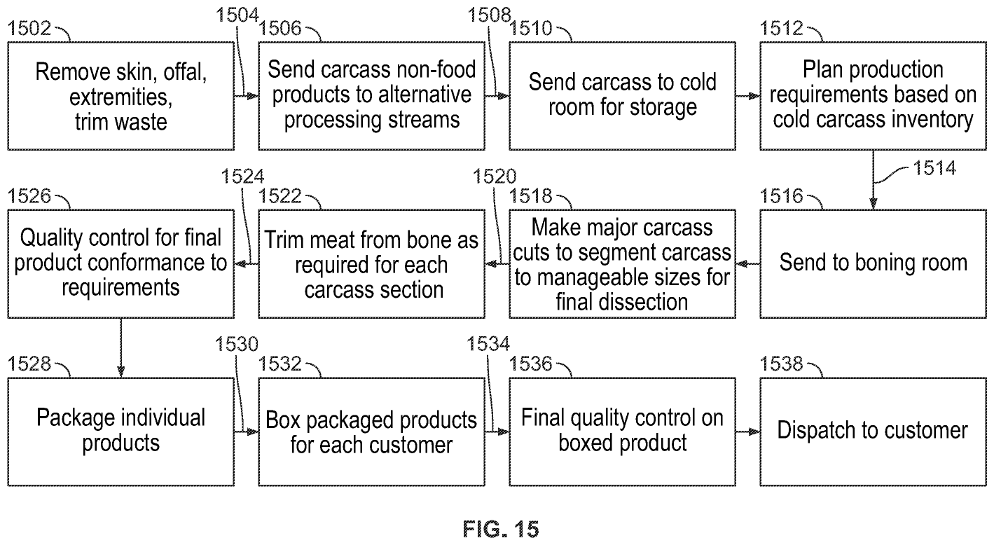

FIG. 15 is a workflow illustrating use of a plurality of 3D X-ray computed tomography scanning processes for improved abattoir management and automation, in accordance with some embodiments of the present specification. In embodiments, a controller in data communication with a 3D X-ray computed tomography scanner implements a plurality of instructions or programmatic code to receive 3D scan image data, process or analyze the scan image data and generate various outputs or outcomes.

At step 1502, an animal is processed to remove skin, offal, extremities and trim waste. At step 1504, full carcass scanning or inspection is conducted while a temperature of the carcass is in a range of 10 to 50 degrees Celsius, and preferably is greater than 10 degrees Celsius, using a 3D X-ray computed tomography scanning system such as those described with reference to FIGS. 9A, 9B, 10A, 10B, 11 and 12. At this stage, the carcass scan data may be analyzed to determine measurements or information related to eating quality, understand animal health, determine carcass value, provide input to the production planning process, and enable optimal processing of the animal to meet customer demand. In various embodiments, a value of the carcass is determined based on at least one of lean meat yield, ratio of intra-muscular fat to tissue, amount of inter-muscular fat, absolute and relative size of individual organs, muscle volume, number of ribs, and presence or absence of cysts, tumors, pleurisy and foreign objects.

Consequently, at step 1506, non-food products of the carcass are sent to alternative processing streams. At step 1508, scanning is conducted of offal and other by-products to provide further input to animal health measurements (for example, inspection of individual organs for abnormalities and presence or absence of cysts, tumors, pleurisy and foreign objects). This can again affect carcass health, carcass valuation and subsequent production process planning. Thereafter, at step 1510, the carcass is sent for storage in a cool room that is maintained at a temperature of less than 15 degrees Celsius and preferably at about 12 degrees Celsius. Production requirements are planned, at step 1512, based on cold carcass inventory.

Now, at step 1514, full scanning of the carcass is conducted once the carcass has been stored in the cold room for a period ranging from 24 to 36 hours. At this point, the carcass will have settled into a rigid shape and re-imaging with the 3D X-ray computed tomography system ensures that the most accurate scan image data, indicative of the bone, fat and tissue structure and, therefore, of areas of contiguous meat of a predefined quality level (determined by, for example, ratio of intra-muscular fat to tissue and amount of inter-muscular fat), is sent to automated cutting systems that are used to do initial carcass segmentation into smaller pieces for more effective processing in a boning room. At step 1516, the carcass is sent to the boning room and thereafter, at step 1518, the automated cutting systems perform major carcass cuts to segment the carcass to manageable sizes for final dissection.

Next, at step 1520, in some embodiments, a 3D X-ray screening system with smaller inspection area, aperture, tunnel or region (such as that of the screening system of FIG. 4) is used to scan the smaller carcass sections (resulting from step 1518) in order to generate scan image data and determine therefrom accurate 3D structures of the smaller carcass sections prior to automated de-boning of expensive cuts, such as a beef strip-loin. Here, accurate registration between the 3D scan image data and the automated cutting systems is critical to avoid waste of valuable product and bone chipping into the final product. Subsequently, at step 1522, meat is trimmed from the bone as required from each smaller carcass section.

At step 1524, in some embodiments, the 3D X-ray screening system with smaller inspection area, aperture, tunnel or region is used to scan the meat and the scan image data is analyzed to determine measurements related to individual dissected cuts, such as a T-bone or rib-eye steak, for key quality metrics such as eating quality, fat thickness and presence of foreign objects including bone fragments. The amount of meat remaining on the bone after de-boning is also determined. If excess meat remains, the bone may be sent back for further processing to extract the remaining meat into the food chain. Subsequently, at step 1526, a quality control function is performed to ensure final product conformance to customer requirements and then, at step 1528, individual meat products are packaged.

Next, at step 1530, a quality control scanning is performed of individual cuts following packaging. This inspection is targeted towards looking for foreign objects as well as for measures such as fat thickness surrounding a piece of steak, for example, in order to ensure that customer requirements have been met. In some embodiments, this step is done with a 3D X-Ray CT system (e.g. FIG. 12), a two-dimensional X-ray system or a camera system. Packaged meat products are now boxed, at step 1532, for each customer.

Now, at step 1534, an entire box of packaged meat is scanned through the 3D X-ray computed tomography system with a smaller inspection area, aperture, tunnel or region to facilitate a final quality control function. During the final quality control function, at step 1536, a packing list to be given to the customer is compared against the actual contents of the box using automated analysis methods, such as deep learning methods, for example, to validate that the correct number of each type of product are in the box with the desired eating quality, shape and size specifications wherein the eating quality is determined based on at least one of a ratio of intra-muscular fat to tissue and an amount of inter-muscular fat. Finally, at step 1538, the boxed product is dispatched to the customer.

In embodiments, steps 1504, 1508, 1514, 1520, 1524, 1530 and 1534 highlight processes where 3D X-ray carcass inspection adds value to improving overall abattoir production operation.

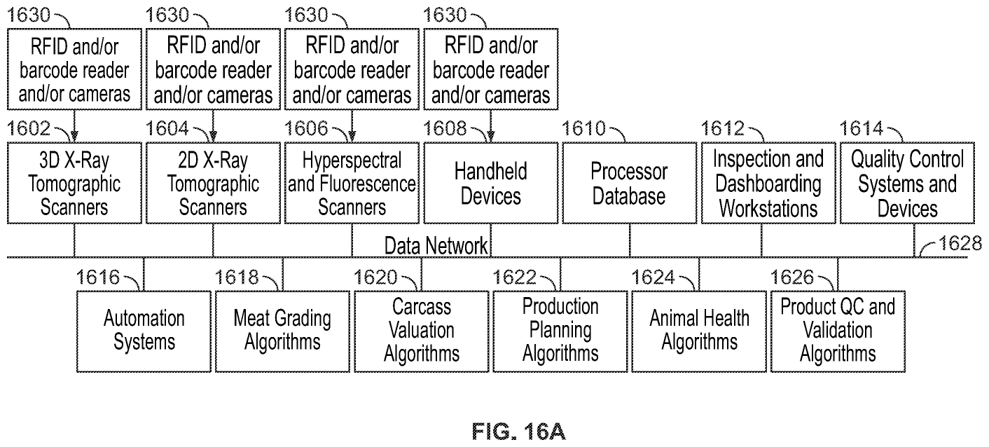

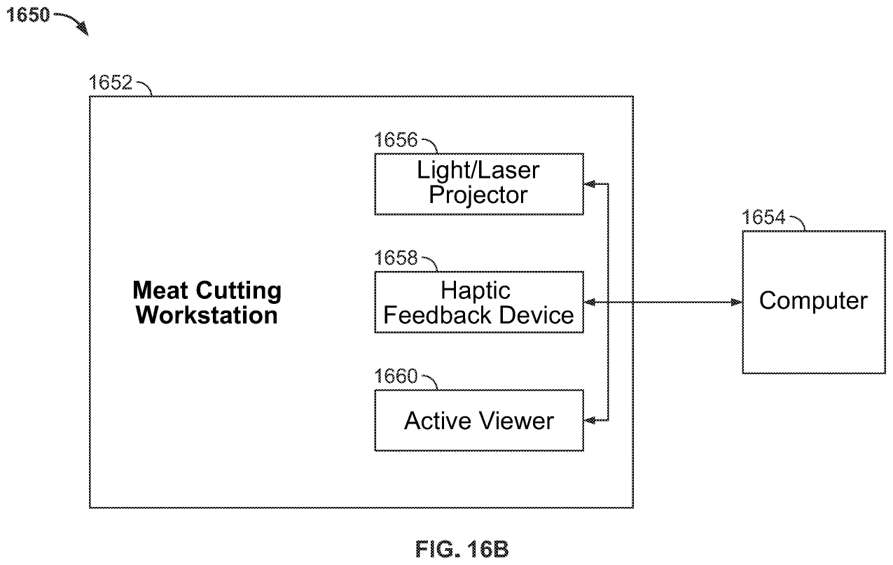

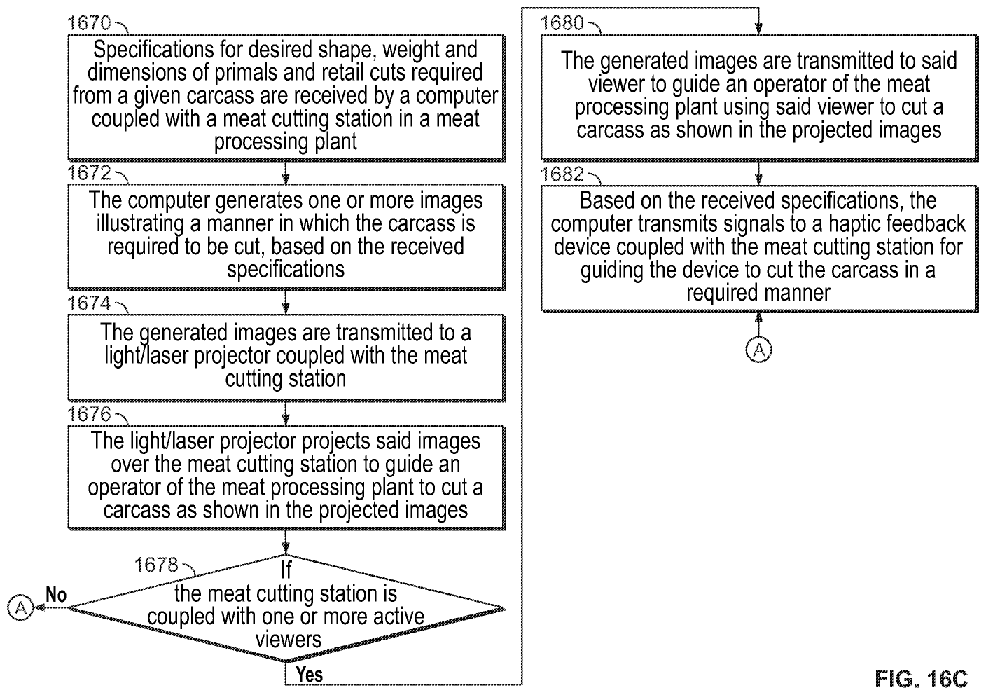

FIG. 16A is a workflow diagram illustrating an exemplary networked layout of a semi-automated meat production process, in accordance with an embodiment of the present specification. In an embodiment, meat production process workflow 1600 comprises 3D X-Ray tomographic scanners 1602; 2D X-Ray tomographic scanners 1604; hyperspectral and fluorescence scanners 1606; handheld devices 1608; database 1610; inspection workstations 1612; quality control systems and devices 1614; automation systems 1616; meat grading algorithms 1618; carcass valuation algorithms 1620; production planning algorithms 1622; animal health algorithms 1624; and product quality check and validation algorithms 1626, wherein all element blocks are coupled to a common communications/data network 1628. In embodiments, the meat production process also comprises 3D and 2D X-Ray scanners and other sensing elements such as RFID and/or barcode reader and/or cameras 1630.

In embodiments, the common communications/data network 1628 enables storage and retrieval of data in real-time from the database 1610 thereby providing a rapid search facility in order to store and retrieve data.

The common communications/data network 1628 also facilitates transmission of image data from the sensing elements (such as, but not limited to, the 3D X-Ray tomographic scanners 1602, the 2D X-Ray tomographic scanners 1604, the hyperspectral and fluorescence scanners 1606, and the handheld devices 1608), in real-time, to the algorithm processing units that can analyze the data from said sensing elements to generate information required for optimal operation of the meat production process.

The common communications/data network 1628 also enables the data from the sensing elements to be passed in real-time to automated cutting systems employed in the meat production process as well as to human operators to direct cutting of carcasses and/or primals into retail cuts on a carcass-by-carcass basis. The common communications/data network 1628 also enables the data from the sensing elements employed in the meat processing plant to be analyzed by automated quality control processes 1626 and human quality control staff to ensure accurate processing and food safety standards. In an embodiment, the common communications/data network 1628 provides means for real-time display of production metrics and other data (such as financial reports) that support meat production plant management in delivering the highest possible productivity from the plant.

Referring to FIG. 16A, in an embodiment, the present specification provides inspection workstations 1612 which operates as a plant management dashboard providing an operator of a meat processing plant with real-time updates of the status of all products within the plant. In embodiments, said status information comprises: real-time location of a carcass, primal, retail-cut, trim or packaged product identified by means of a unique ID. In an exemplary scenario, if a sensing element such as one or more of the 3D X-Ray tomography scanners 1602, 2D X-Ray tomography scanners 1604, hyperspectral and fluorescence scanners 1606, handheld devices 1608, detects fecal contamination of a particular primal through its unique ID, the inspection workstations/dashboard 1612 immediately displays, for an operator to view, a location of the remaining carcass, and any other primals, retail cuts, trim or packaged product that originated from the same carcass.

In an embodiment, status information displayed by the inspection workstations/dashboard 1612 comprises at least one of: real-time notification of any package mis-labelling or incorrect shippable carton contents; real-time notification and location of any animal health defects identified by any sensing element or human operator within the plant; real-time production data including output over adjustable time scales (e.g. current shift, day, week, month or year); real-time plan variance; real-time notification of areas of production backlog or product non-conformity that require management action; real-time financial data on retail product value based on objective measurement from suitable sensors within the plant; and other relevant data such as, but not limited to, staff utilization, staff efficiency and work accuracy.

In an embodiment, the present specification provides a method of identifying the locations of all staff working in a meat processing plant in real time, by providing each member of the staff with Wi-Fi, GPS or other suitable location sensors. Referring to FIG. 16A, in embodiments, video camera systems are installed in the premises of the meat processing plant for providing real-time data to analysis algorithms such as meat grading algorithms 1618, carcass valuation algorithms 1620, production planning algorithms 1622, animal health algorithms 1624, and product quality check and validation algorithms 1626. In embodiments, said real time data is used for conducting: automated time and motion studies to determine where plant efficiencies may be achieved by more productive use of people and facilities; automated technique analysis to determine distinguishing characteristics of high performing operators which may then be used for training low performing/less efficient operators; automated review of safe working practices for all staff working with knives to determine best practice to enhance overall plant safety; and quality assurance.

In an embodiment, the present specification provides an augmented reality based method for achieving optimal cutting of carcasses, primals and retail cuts in a meat processing plant. FIG. 16B is a block diagram illustrating an augmented reality based system for cutting meat in a meat processing plant. In an embodiment, system 1650, located in a meat processing plant, comprises a meat cutting station 1652 for cutting carcasses into both primals and retail cuts. The meat cutting station 1652 is coupled with a controller workstation 1654. In an embodiment, the meat cutting station 1652 comprises a light/laser projector 1656, one or more haptic feedback devices 1658, and one or more active viewers 1660 for electronically guiding an operator to cut the carcasses in a desired manner. Each of the light/laser projector 1656, the one or more haptic feedback devices 1658, and the one or more viewers 1660 are electronically coupled with the controller workstation 1654, which, in an embodiment, is a computing device that controls the operation of said devices. In an embodiment, the light/laser projector 1656 is used to project images of desired primal and retail cuts over the meat cutting station 1652 to guide an operator of the meat processing plant to cut a carcass as shown in the projected images. In another embodiment, the one or more haptic feedback devices 1658 comprise cutting tools such as, but not limited to a knife blade haptic that stops vibrating when the knife is in a desired position with respect to a carcass for enabling an operator to produce desired primal and retail cuts from a carcass. In an embodiment, the one or more viewers 1660 comprise wearable active glasses to project or otherwise display how and where an individual carcass, primal or retail cut should be cut or trimmed to deliver optimal results. It would be apparent to persons of skill in the art that other feedback mechanisms such as, but not limited to, audible tones, indicators or video monitors may also be used either solely or in conjunction with other augmented reality devices for enabling an operator to deliver an optimal cut.

FIG. 16C is a flowchart illustrating the steps of an augmented reality-based method for cutting meat in a meat processing plant. At step 1670, specifications for desired shape, weight and dimensions of primals and retail cuts required from a given carcass are received by a computer coupled with a meat cutting station in a meat processing plant. At step 1672 the computer generates one or more images illustrating a manner in which the carcass is required to be cut, based on the received specifications. In embodiments, said images include locations and angles of cuts required with respect to different parts of the carcass. At step 1674 the generated images are transmitted to a light/laser projector coupled with the meat cutting station. At step 1676 the light/laser projector projects said images over the meat cutting station to guide an operator of the meat processing plant to cut a carcass as shown in the projected images. At step 1678, the computer determines if the meat cutting station is coupled with one or more active viewers, which in an embodiment comprise wearable active glasses to project or otherwise display how and where an individual carcass, primal or retail cut should be cut or trimmed to deliver optimal results. At step 1680 if the meat cutting station is coupled with one or more active viewers, the generated images are transmitted to said viewer to guide an operator of the meat processing plant using said viewer to cut a carcass as shown in the projected images. At step 1682 based on the received specifications, the computer transmits signals to a haptic feedback device coupled with the meat cutting station for guiding the device to cut the carcass in a required manner. In an embodiment, haptic feedback devices comprise cutting tools such as, but not limited to a knife blade haptic that stops vibrating when the knife is in a desired position with respect to a carcass for enabling an operator to produce desired primal and retail cuts from a carcass.

Referring to FIG. 16A, in various embodiments, data produced by all sensor systems such as, but not limited to, the 3D X-Ray tomography scanners 1602, 2D X-Ray tomography scanners 1604, hyperspectral and fluorescence scanners 1606, and handheld devices 1608 installed in a meat processing plant is analyzed by automated algorithms such as meat grading algorithms 1618, carcass valuation algorithms 1620, production planning algorithms 1622, animal health algorithms 1624, product quality check and validation algorithms 1626 to produce information that is used to drive the meat production process.

In an embodiment, the automated, real-time, carcass valuation algorithms 1620 identify a carcass/an item derived from a carcass as being contaminated (for example, by using hyperspectral and fluorescence scanners 1606). Carcass valuation algorithms 1620 also identify the products (primals and cuts) derived from the same carcass as the contaminated item and marks all such products for de-contamination or further analysis depending on a type of contamination.

In an embodiment, the automated, real-time, carcass valuation algorithms 1620 also identify health defects in carcasses, animal offal, and primals. For example, pleurisy; metal contamination from sources such as, but not limited to, fence wire or syringe needles; tumors or cysts may be identified in carcasses. In addition, tumors, cysts, enlarged organs, and worms may be identified in offal by using for example using hyperspectral and fluorescence scanners 1606 and 3D X-Ray tomography scanners 1602, 2D X-Ray tomography scanners 1604. Further, worm nodules, tumors and cysts may be identified in primals; and discoloration, worms, tumors, and cysts may be identified in retail cuts being processed in the meat processing plant by using for example 3D X-ray tomographic imaging. In another embodiment, the automated, real-time, carcass valuation algorithms 1620 also identifies 3D spatial location of bone structure, muscles, inter-muscular fat or health defects within carcasses and primals in order to drive automated cutting equipment and to direct human operators, for example, by using 3D X-ray computed tomography image sensors.

In an embodiment, the automated, real-time product quality check and validation algorithms 1626 identifies meat quality spatially distributed within a carcass, primal or retail cut or packaged product against suitable grading standards such as the Australian MSA standard or the USDA meat quality standard by using imaging data obtained from sensing devices employed in the meat processing plant, such as, but not limited to 3D X-Ray tomography scanners 1602, 2D X-Ray tomography scanners 1604, hyperspectral and fluorescence scanners 1606, and handheld devices 1608.

Further, in an embodiment, the automated, real-time, carcass production planning algorithms 1622 performs carcass valuation, including determining optimal ways to cut the carcass to maximize product revenue given the current customer product delivery requirements. In an embodiment, production planning algorithms 1622 operates by combining objective measurement data derived from sensor systems such as the 3D X-Ray tomography scanners 1602, 2D X-Ray tomography scanners 1604, hyperspectral and fluorescence scanners 1606, and handheld devices 1608 installed in the meat processing plant including spatially localized information on meat grading, muscle volume, animal health, number of ribs in the carcass and animal health data obtained via the meat grading algorithms 1618, carcass valuation algorithms 1620, and the animal health algorithms 1624.

In an embodiment, real-time, meat grading algorithms 1618 determines the constituents of trim boxes to determine the exact ratio of fat to lean meat. In an embodiment data from sensing elements such as 3D X-ray tomography system employed in the meat producing plant is used by the meat grading algorithms 1618 to generate metrics for both percentage fraction of fat and lean as well as the size distribution of lean and fat items within the trim box.

In an embodiment, real-time, product quality check and validation algorithms 1626 determine if the labelling of packaged retail cuts is done as per predefined rules. In an embodiment, data from sensing elements such as 3D X-ray tomography system in combination with hyperspectral imaging employed in the meat producing plant is used to determine the weight, meat grade, meat color, fat content, fat thickness and cut-type of the products produced at the plant.

In an embodiment, real-time, product quality check and validation algorithms 1626 also determines if the contents of cartons containing multiple packaged retail cuts is as per predefined customer requirements. In an embodiment data from sensing elements such as 3D X-ray tomography system 1602 employed in the meat producing plant is used by the product quality check and validation algorithms 1626 to determine parameters such as cut type, meat grading score, weight and fat thickness of each retail cut within the carton, which parameters are then compared to the customer supplied product requirements obtained from the production database 1610. In an embodiment, real-time, product quality check and validation algorithms 1626 also performs automated tracking of product throughout the plant by using sensing technology such as, but not limited to, RFID, barcode, video tracking and time, velocity and distance based methods.

In embodiments, real-time data analysis algorithms provided by the present specification also perform time and motion analysis of individual operators and groups of operators based on video camera and location sensor measurements throughout a meat processing plant. It would be apparent to persons skilled in the art that other automated analysis algorithms may also be employed in meat processing plant. Examples of some such real time automated algorithms comprise algorithms for monitoring temperature distribution, humidity variation, throughput and other associated production metrics such as touch labor time per carcass, and the examples of real time analysis algorithms provided herein are for representative purpose only and should not be considered limiting the scope of the present specification.

Referring to FIG. 16A, the present specification provides for the use of image sensors such as 3D X-ray tomographic scanners 1602, which have dual or multi-energy sensors placed in a rotating gantry being robotically controlled or stationary gantry imaging geometries. In embodiments, 3D X-ray tomographic scanners 1602 may be used with or without motion correction methods depending upon the application in which the scanning technology is deployed. In various embodiments, the 3D X-ray tomographic scanners 1602 may be used in various applications in a meat producing plant. In an embodiment said scanner is used for performing full carcass scanning while the carcass is still hot on entrance to the abattoir production line post slaughter in the meat processing plant, wherein the scanner obtains data comprising carcass volume, spatially localized meat quality grading, bone structure and initial cutting line analysis, gross animal health defects including metal object inclusion, tumors and cysts, and wherein the data is used for obtaining an accurate carcass valuation and retail revenue estimate. In an embodiment, the 3D X-ray tomographic scanner 1602 is used for performing full carcass scanning once the carcass is rigid after cooling for one or two days wherein the scanner obtains data to map the 3D carcass structure to a sub-millimeter precision in order to determine final cut lines for automatic or manual processing into primals. In an embodiment, said scanner is used for performing offal screening in order to determine presence of cysts, tumors, metal and other foreign objects and subsequent analysis for abnormal organ volume and densities. In another embodiment, the 3D X-ray tomographic scanner 1602 is used for performing primal scanning for determining a sub-millimeter 3D location of carcass features immediately prior to automated or manual cutting equipment in the boning room. During such scanning, in some embodiments, the primal is fixed to rigid support structures that may be used to transfer the primal from the imaging system (scanners 1602) to an automated, robotic, cutting equipment in a known frame of reference in the meat processing plant. In an embodiment, the 3D X-ray tomographic scanner 1602 is used for performing retail cut scanning to determine a cut type, a meat grade, a weight, a fat thickness and an orientation of a cut within a package. The obtained scanned data may then be used to cross-correlate with the label applied to the package using optical character recognition technology taken from a video camera image or a bar code reader. In another embodiment, the scanned data may be used to auto generate an accurate label which may then be applied directly to the packaged retail cut. In an embodiment, the 3D X-ray tomographic scanner 1602 is used for scanning packaged carton in order to verify that the entire contents of the carton containing multiple retail cuts reflects accurately the label that is applied to the outside of the carton. In embodiments, each retail cut within the carton is analyzed from the obtained 3D X-ray image in order to determine a cut type, a meat grade, a weight, a fat-thickness and a 3D location of each retail cut within the carton.

Referring to FIG. 16A, the present specification provides for the use of image sensors such as 2D X-ray tomographic scanners 1604, which have dual or multi-energy sensors placed in a rotating gantry being robotically controlled or stationary gantry imaging geometries. In embodiments, 2D X-ray tomographic scanners 1604 may be used with or without motion correction methods depending upon the application in which the scanning technology is deployed. In various embodiments, the 2D X-ray tomographic scanners 1604 may be used in various applications in a meat producing plant. In an embodiment, said scanner is used for performing meat grading scans, typically for beef grading; wherein the X-ray scan data is analyzed for checking meat quality, rib-eye muscle area, inter-muscular fat thickness, intra-muscular fat content, fat marbling and surface fat thickness. In an embodiment, the 2D X-ray tomographic scanner 1604 is used for performing analysis of a section through a trim carton to determine average fat to lean ratio across a retail cut slice and average trim component dimensions (both fat and lean) within the slice.

The present specification also provides for the use of image sensors such as 2D projection X-ray imaging in single-view or dual-view configurations with dual or multi-energy X-ray sensors. In various embodiments, the 2D projection X-ray imaging may be used in various applications in a meat producing plant. In an embodiment, said imaging is used for performing analysis of offal after removal from the carcass into trays, wherein one tray of green offal (e.g. stomach, intestines and bowel) and one tray of red offal (e.g. heart, lungs, liver, kidneys) are produced per carcass. In embodiments, the X-ray system is used to look for foreign objects such as metal items and worm nodules as well as for health defects such as tumors, cysts and enlarged organs. In an embodiment, said imaging is also used for analysis of cartons containing trim to determine the fraction of lean to fat tissue averaged over the whole carton.

Referring to FIG. 16A, the present specification provides for the use of hyperspectral and fluorescence scanners 1606 operating across the mid infra-red wavelengths ranging from 5,000 nm to 2,000 nm; short wave infra-red wavelengths ranging from 2,000 nm to 900 nm; near infra-red wavelengths ranging from 900 nm to 800 nm; visible light wavelengths ranging from 800 μm to 400 nm; and ultra-violet wavelength ranging from 400 nm to 100 nm. Contrast between tissues varies as a function of wavelength, as a function of healthy to diseased tissue and as a function of contaminated to clean tissue. In embodiments, ultra-violet light, broadband visible light and infrared light may be used to illuminate offal/other meat product under inspection for reflective image formation and analysis. In embodiments, hyperspectral and fluorescence scanners 1606 may be used for carcass scale analysis of contamination (for example, for detection of feces following removal of the hide). In embodiments, hyperspectral and fluorescence scanners 1606 may be used for analysis of viscera (offal) following removal from the carcass into trays, which typically includes one tray of green offal (such as stomach, intestines and bowel) and one tray of red offal (such as heart, lungs, liver, kidneys) per carcass. In embodiments, hyperspectral and fluorescence scanners 1606 are used to determine a range of heath defects in meat products such as, but not limited to tumors, cysts, inflammation, rashes and infection.

Referring to FIG. 16A, the present specification provides for the use of video camera systems 1630 operating in the visible wavelength ranging from 800 nm to 400 nm and short wave infra-red wavelength region ranging from 2000 nm to 900 nm. In embodiments, said video camera systems are used for: object tracking along conveyor belts and between hanging conveyor systems, automated cutting systems and horizontal conveyor systems; positioning data for locating carcasses and primals within other inspection systems such as X-ray scanners and to provide data for motion correction algorithms as may be required; human factors and time-motion study analysis to deliver optimal production efficiency and best quality operator cutting procedures; and thermal imaging to analyze knife cutting methods used in creating retail cuts from primals.

Referring to FIG. 16A, the present specification provides for the use of handheld devices 1608 comprising RFID and/or barcode reader and/or cameras 1630. In various embodiments, said RFID and/or barcode reader and/or cameras 1630 which may be both handheld or fixed are used for: tracking carcass, offal, primal, retail cut, trim container, packaged product and cartons or product within the production facility; and performing quick lookup of data relating to the carcass, offal, primal, retail cut, trim container, packaged product or carton of product associated with that barcode and/or RFID tag reading.

In various embodiments, various different types of sensors and applications may be used in the abattoir, of a meat processing plant, such as, but not limited to, fixed installations of 3D video camera systems; radar range finding systems for determining carcass volume, meat grading and meat color; hand held systems for measuring temperature, pH, color, contamination and other parameters. Such and other sensors may be integrated within the overall framework disclosed in the present specification for further increasing the efficiency and profitability of a meat processing plant, without departing from the scope of the present specification.

In an embodiment of the present specification, each of the carcasses being processed in a meat processing plant, each of the primals that are cut from said carcasses and each subsequent retail cut from each of said primals are provided with a unique identifier (ID) to ensure traceability of all products. For example, if a carcass entering an abattoir cool room of the meat processing plant has an ID of ‘63’, and subsequently, six primals are cut from the carcass, said primals may be provided with ID's such as, ‘63:1’ through ‘63:6’. If the primal ‘63:1’ is then processed into 26 retail cuts said cuts may be provided with ID's such as ‘63:1:1’ to ‘63:1:26’. If the primal 63:2 is processed into 15 retail cuts said cuts may be provided with ID's such as ‘63:2:1’ to ‘63:2:15’. IDs for the retail cuts from the remaining primals from carcass ID ‘63’ may be similarly provided. It would be apparent to persons of skill in the art, that multiple carcass, primal and retail cut labelling schemes are possible and may be employed in the present specification, and that the above given example is just one of such labelling schemes. In various embodiments, the IDs generated for the carcass, primal and retail cut are also associated with the date and time stamp at which a primal was cut from a carcass or a retail cut was separated from its primal.

In another embodiment, the present specification provides a method for tracking a location and time or arrival of each carcass, primal and retail cut through a meat processing plant.

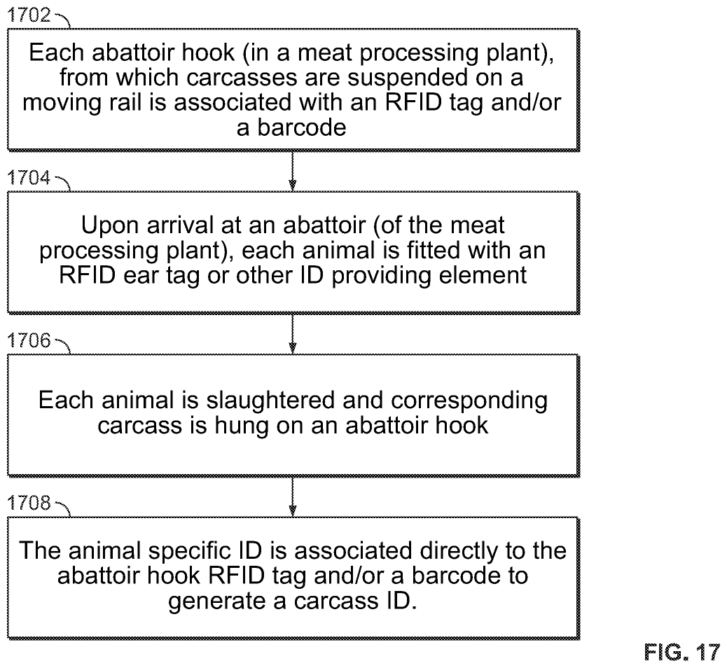

FIG. 17 is a flowchart illustrating the steps of assigning a carcass ID for tracking a location and time or arrival of each carcass through a meat processing plant, in accordance with an embodiment of the present specification. At step 1702 each abattoir hook (in a meat processing plant), from which carcasses are suspended on a moving rail is associated with an RFID tag and/or a barcode. At step 1704, upon arrival at an abattoir (of the meat processing plant), each animal is fitted with an RFID ear tag or other ID providing element. At step 1706 each animal is slaughtered and corresponding carcass is hung on an abattoir hook. At step 1708, the animal specific ID is associated directly to the abattoir hook RFID tag and/or a barcode to generate a carcass ID. This ensures traceability of an animal to a corresponding carcass. In embodiments, a carcass ID is a combination of the abattoir hook ID and the animal ID to ease traceability of the carcass primary ID back to a farm on which the animal was produced. In embodiments, a date and time stamp is included as a component of the carcass ID to ease human sortation of the abattoir data.

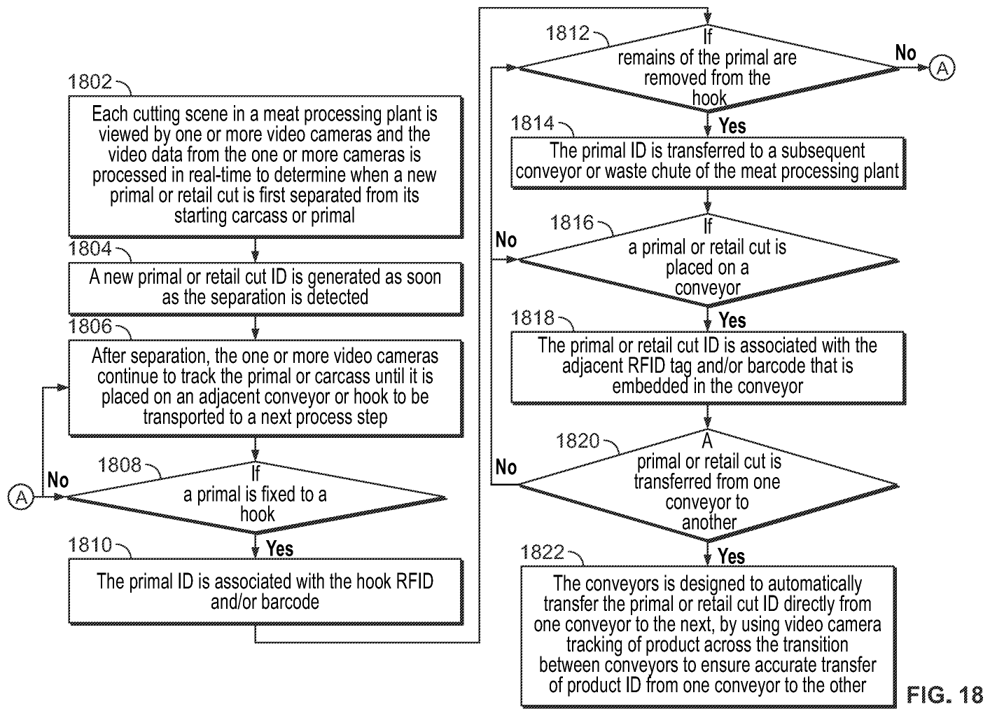

In an embodiment, the present specification employs a video camera technology to track a primal as it is cut from a carcass and transferred to a conveyor or a secondary hanging rail. FIG. 18 is a flowchart illustrating the steps of assigning a carcass ID for tracking a location and time when a primal or a retail cut is obtained from a carcass through a meat processing plant, in accordance with an embodiment of the present specification. At step 1802, each cutting scene in a meat processing plant is viewed by one or more video cameras and the video data from the one or more cameras is processed in real-time to determine when a new primal or retail cut is first separated from its starting carcass or primal. At step 1804, a new primal or retail cut ID is generated as soon as the separation is detected. At step 1806, after separation, the one or more video cameras continue to track the primal or carcass until it is placed on an adjacent conveyor or hook to be transported to a next process step. At step 1808 it is determined if a primal is fixed to a hook. At step 1810, if a primal is fixed to a hook, the primal ID is associated with the hook RFID and/or barcode. At step 1812 it is determined if remains of the primal are removed from the hook. At step 1814 if remains of the primal are removed from the hook, the primal ID is transferred to a subsequent conveyor or waste chute of the meat processing plant. At step, 1816 it is determined if a primal or retail cut is placed on a conveyor. At step 1818, if a primal or retail cut is placed on a conveyor, the primal or retail cut ID is associated with the adjacent RFID tag and/or barcode that is embedded in the conveyor. In an embodiment, conveyor ID's are placed at a spacing ranging from 100 mm-200 mm on the conveyor so that the position of each primal or retail cut on the conveyor is easily identifiable. At step, 1820 it is determined if a primal or retail cut is transferred from one conveyor to another. At step 1822, if a primal or retail cut is transferred from one conveyor to another, the conveyors are designed to automatically transfer the primal or retail cut ID directly from one conveyor to the next, by using video camera tracking of product across the transition between conveyors to ensure accurate transfer of product ID from one conveyor to the other. In an embodiment, if more than one retail cut is placed side by side on a conveyor, such that a plurality of cuts are associated with the same conveyor barcode or RFID tag, video camera tracking is used to determine the lateral position of each cut on the conveyor at the point where the cuts are loaded or removed from the conveyor.

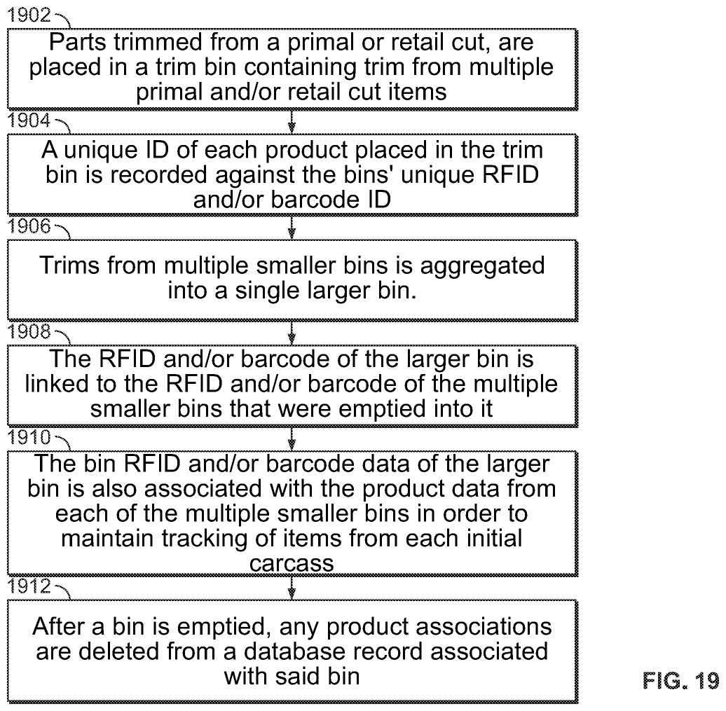

In embodiments, for the points where human operators lift or otherwise remove primals or product from a rail or conveyor into a subsequent processing step, such as trimming fat from a primal or packing the product, one or more video cameras are used to monitor the location of the product and any parts that may be cut from it in order to maintain product location and ID assurance. FIG. 19 is a flowchart illustrating the steps of assigning a carcass ID for tracking a location of a carcass/primal/retail cut removed by human operators through a meat processing plant, in accordance with an embodiment of the present specification. At step 1902 parts trimmed from a primal or retail cut, are placed in a trim bin containing trim from multiple primal and/or retail cut items. At step 1904 a unique ID of each product placed in the trim bin is recorded against the bins' unique RFID and/or barcode ID. At step 1906, trims from multiple smaller bins are aggregated into a single larger bin. At step 1908, the RFID and/or barcode of the larger bin is linked to the RFID and/or barcode of the multiple smaller bins that were emptied into it. At step 1910, the bin RFID and/or barcode data of the larger bin is also associated with the product data from each of the multiple smaller bins in order to maintain tracking of items from each initial carcass. At step 1912 after a bin is emptied, any product associations are deleted from a database record associated with said bin in order that when the bin is filled again it may be associated with corresponding new product IDs.

In embodiments, before and after photographic data is recorded and associated with an initial and final product for quality assurance purposes at points where automated process equipment, such as rotating blades, band saws, pulling devices or water jet cutters, removes or modifies carcass, primal or retail cuts. Where automated handling equipment moves carcasses, primals or retail cuts from one location to another, the carcass, primal or retail cut IDs are transferred automatically from the initial location to the final hook or conveyor location.

In embodiments, at each point in the meat processing plant, where a carcass, primal, retail cut or a packaged product is scanned by a sensor, the carcass, primal, retail cut or packaged product ID is associated directly with the data produced by the sensor to allow instant recall of the data from that sensor via the data network (such as 1628, FIG. 16A) for retrospective analysis and for real-time analysis by computerized algorithms that provide added value to the overall production process.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A stationary gantry X-ray computed tomography (CT) imaging system adapted to scan an animal in a farm, wherein the CT imaging system is housed in a first enclosure and surrounded with at least one second enclosure and comprises:
a horizontal platform configured to direct the animal through a scanning area for inspection;
a first plurality of X-ray sources positioned at least partially around the scanning area to scan the animal in a first imaging plane;
a first array of detectors, wherein the first array of detectors is offset from the associated first plurality of X-ray sources such that X-rays from each of the first plurality of X-ray sources on one side of the scanning area interact with corresponding each of the first array of detectors on an opposing side of the scanning area to form a first transmission image data of the animal;
a second plurality of X-ray sources positioned at least partially around the scanning area to scan the animal in a second imaging plane;
a second array of detectors, wherein the second array of detectors is offset from the associated second plurality of X-ray sources such that X-rays from each of the second plurality of X-ray sources on one side of the scanning area interact with corresponding each of the second array of detectors on an opposing side of the scanning area to form a second transmission image data of the animal;
a controller configured to control an activation and deactivation of each of the first plurality of X-ray sources and each of the second plurality of X-ray sources; and
at least one workstation configured to receive and process the first and second transmission image data and generate a three-dimensional image of the animal as the animal passes through the scanning area.

2. The system of claim 1, wherein the scanning area has a substantially rectangular geometry and wherein a value representative of an entire width of the scanning area is within 85% of a value representative of an entire length of the scanning area.

3. The system of claim 1, wherein the first and second imaging planes are disposed along a direction perpendicular to the direction of motion of the animal over the horizontal platform.

4. The system of claim 1, wherein the first plurality of X-ray sources are offset from the associated first array of detectors, in the first imaging plane, by a first distance, wherein the second plurality of X-ray sources are offset from the associated second array of detectors, in the second imaging plane, by a second distance, and wherein the first distance is equal to the second distance and ranges from 2 mm to 20 mm.

5. The system of claim 1, wherein the first imaging plane comprises four X-ray sources separated from each other by gaps, and wherein the second imaging plane comprises four X-ray sources positioned to align with the gaps.

6. The system of claim 1, wherein the first and second imaging planes are separated by a distance ranging from 200 mm to 2000 mm.

7. The system of claim 1, wherein the first plurality of X-ray sources and the second plurality of X-ray sources comprise linear multi-focus X-ray sources, and wherein the controller is configured to switch on each source point within a first of the linear multi-focus X-ray sources and subsequently switch on each source point within a second of the linear multi-focus X-ray sources that is not adjacent to the first linear multi-focus X-ray source.

8. The system of claim 7, wherein the second linear multi-focus X-ray source is 20 to 90 degrees away from the source point within the first linear multi-focus X-ray source.

9. A stationary gantry X-ray CT imaging system to scan an animal in a farm, comprising:
a horizontal platform configured to enable the animal to pass through a scanning area for inspection;
a plurality of X-ray sources disposed in a plane at least partially around the scanning area;
an array of detectors deployed at least partially around the scanning area to form transmission scan data of the animal;
a controller configured to control an activation and deactivation of each of the plurality of X-ray sources; and
at least one workstation configured to received and process the transmission scan data and to determine at least one of lean meat yield, ratio of intra-muscular fat to tissue, amount of inter-muscular fat, absolute and relative size of individual organs, and presence of cysts, tumors, pleurisy and foreign objects corresponding to the animal.

10. The system of claim 9, wherein the plurality of X-ray sources comprises 200 to 500 X-ray source emission points around an anode and wherein each of the X-ray source emission points and the anode are enclosed in a vacuum tube.

11. The system of claim 10, wherein each of the X-ray source emission points are characterized by a tube voltage in a range of 120 kV to 200 kV and a tube current in a range 1 mA to 20 mA.

12. The system of claim 9, wherein each of the plurality of the X-ray sources is configured to be operated at a tube voltage of 160 kV and at a tube current of 4 mA.

13. The system of claim 12, wherein each of the plurality of the X-ray sources is operated corresponding to total X-ray beam power of 640 W.

14. The system of claim 9, wherein each of the plurality of the X-ray sources comprises an X-ray tube.

15. The system of claim 9, wherein the plurality of X-ray sources are adapted to be operated to deliver a dose per scan to the animal in a range of 2 μSv to 20 μSv.

16. The system of claim 9, wherein each of the plurality of X-ray sources are offset from the array of detectors by a distance ranging from 2 mm to 20 mm.

17. The system of claim 9, further comprising a sensor adapted to monitor a surface profile of the animal and measure a motion of the animal.

18. The system of claim 17, wherein the controller is configured to use the measured motion of the animal to determine where X-ray projections should be back-projected into a three-dimensional reconstructed image volume.

19. The system of claim 9, further comprising a first inclined ramp adapted to enable the animal to pass into the scanning area and a second inclined ramp adapted to enable the animal to pass out of the scanning area.
